(19) United States
(12) Patent Application Publication
(10) Pub. No.: US 2024/0287003 A1
(43) Pub. Date: Aug. 29, 2024

(54) ETHACRYNIC ACID DERIVATIVES AS INHIBITORS OF MPRO PROTEASE AND SARS-COV-2 REPLICATION

(71) Applicant: UNIVERSITE EUROMEDITERRANEENNE DE FES, FES (MA)

(72) Inventors: Saïd EL KAZZOULI, FES (MA); Nadia TOUIL, RABAT (MA); Elmostafa EL FAHIME, N.U RABAT. (MA); Abdelmoula EL ABBOUCHI, FES (MA); Mouhssine HEMLALI, RABAT (MA); Nabil EL BRAHMI, FES (MA); Abdelaziz EL ALAOUI, KENITRA (MA); Salim BOUNOU, FES (MA); Mostapha BOUSMINA, FES (MA)

(21) Appl. No.: 18/560,280

(22) PCT Filed: May 10, 2022
(86) PCT No.: PCT/MA2022/000005

§ 371 Date: Nov. 10, 2023

(30) Foreign Application Priority Data
May 12, 2021 (MA) 53521

(51) Int. Cl. C07D 231/56; A61K 31/165; A61K 31/27; A61K 31/40; A61K 31/404; A61K 31/4184; A61K 31/437; A61K 31/4468; A61K 31/495; A61P 31/14; C07C 235/20; C07C 271/20; C07D 209/08; C07D 211/58; C07D 235/06; C07D 295/205; C07D 471/04

(52) U.S. Cl. CPC C07D 231/56; A61K 31/165; A61K 31/27; A61K 31/40; A61K 31/404; A61K 31/4184; A61K 31/437; A61K 31/4468; A61K 31/495; A61P 31/14; C07C 235/20; C07C 271/20; C07D 209/08; C07D 211/58; C07D 235/06; C07D 295/205; C07D 471/04

(57) ABSTRACT

A novel compound of formulae (I) or (II), to the use thereof as a drug, particularly for the treatment of SARS-COV-2, for the treatment of COVID-19 disease and any diseases related to beta-coronaviruses, and for the in vitro application thereof in order to study the interaction with Mpro protease.

### DESCRIPTION AND STATE OF THE ART

Several coronaviruses can cause respiratory infections ranging from the common cold to more serious diseases such as Middle East Respiratory Syndrome (MERS) and Severe Acute Respiratory Syndrome (SARS). COVID-19 disease or severe acute respiratory syndrome is also caused by a virus in the coronavirus family. This virus has been first discovered in Wuhan in China's Hubeie province and named SARS-COV-2 [Zhu et al, N. Engl. J. Med. 2020, 382, 727-73; Li et al, N. Engl. J. Med. 2020, 382, 1199-1207]. Sequencing of the SARS-COV-2 genome has shown that it is 79.6% identical to SARS-COV [Wu et al, Nature 2020, 579, 265-269; Zhou et al, Nature 2020, 579, 270-273].

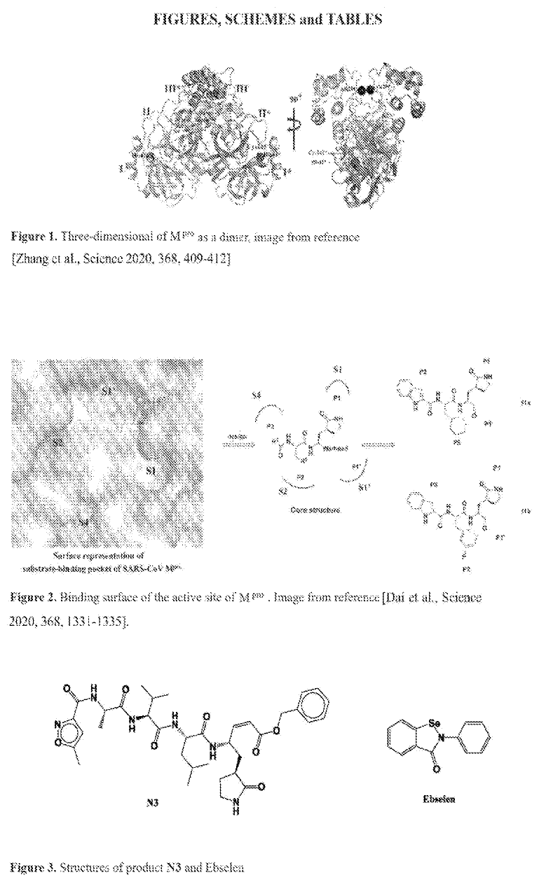

M^{pro} Protease (also known as 3CL^{Pro}) is an enzyme required for the production of proteins which are in turn crucial for the development and replication of the virus. This protease is currently considered to be one of the most promising targets for the development of anti-SARS-COV-2 treatments. This will require the discovery of molecules capable of inhibiting M^{pro}, whose chemical composition and X-ray diffraction structure are now well elucidated [Zhang et al, Science 2020, 368, 409-412]. This enzyme, in the form of a dimer, contains a catalytic site characterised by the presence of two important amino acids, namely His41 and Cys145. In addition, this site is directly involved in the production of amino acids and proteins necessary for the multiplication of the virus [Dai et al., Science 2020, 368, 1331-1335] (FIG. 1).

Following the appearance of the SARS-COV virus in 2002 and 2003, several studies have been carried out around the world to develop small antiviral molecules capable of inhibiting M^{pro}. It should be noted that this protease was also present in SARS-COV and that its structure has not changed much compared with that discovered last year in SARS-COV-2 [Zhang et al., Science 2020, 368, 409-412]). It should also be noted that M^{pro}has no homolog in humans, which is another reason why it is a prime target for the development of antiviral drugs [Kim et al., PLOS Pathog. 2016, 12, e1005531; Yang et al., PLOS Biol. 2005, 3, e324]. In addition, M^{pro} is conserved in several β-coronaviruses (MERS, SARS-COV, SARS-COV-2) which could contemplate the development of a treatment for the current disease but also for future diseases in the event of other health crises caused by other forms of β-coronavirus.

Since the start of the health crisis at the end of 2019, some very interesting works on the inhibition of the SARS-COV-2 M^{pro} protease by small molecules are published in very prestigious journals [Jin et al, Nature 2020, 582, 289-293; Zhang et al, Science 2020, 368, 409-412; Dai et al, Science 2020, 368, 1331-135; Jin et al, Nat. Struct. Mol. Biol. 2020, 27, 529-532]. Following the analysis of these different works, several important and crucial informations for the design and development of anti-SARS-COV-2 are summarised in the following section.

FIG. 1 shows the three-dimensional structure of M^{pro} in two different views, following a 90° rotation. This figure also shows the location of the catalytic site comprised of His41 (blue ball) and Cys145 (yellow ball) and the structure as a dimer of the enzyme.

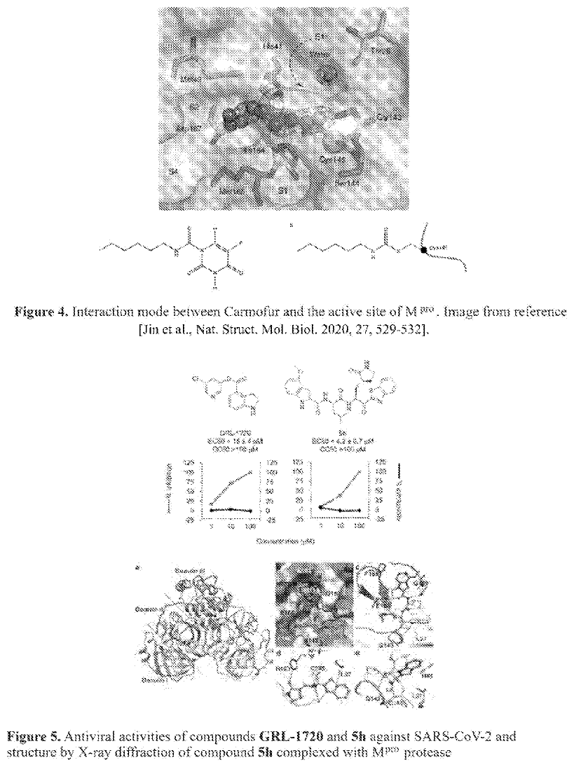

FIG. 2 contains a representation of the binding surface between the M^{pro}active site and ligands (active substances). By virtue of this recent study, teams of Dai, Zhang, Jiang and Su have provided the two molecules 11a and 11b (FIG. 2), each containing an aldehyde covalently binding with Cys145, cyclohexyl or 3-fluorophenyl to occupy pocket S2, and indole for hydrogen bonding with pocket S4 [Dai et al., Science 2020, 368, 1331-1335]. The IC5os (half-maximal inhibitory concentration) of compounds 11a and 11b are 0.053 and 0.040 μM respectively.

Based on the mechanism of interaction between the peptidomimetic compound N3 (FIG. 3) and M^{pro}, Jiang, Rao and Yang identified an inhibitor using computer-aided drug design and then determined the crystal structure of SARS-COV-2 M^{pro}complexed with compound N3. By virtue of high-throughput virtual screening, more than 10,000 compounds have been analysed—including approved drugs, drug candidates in clinical trials and other biologically active compounds. As a result of this study, six compounds have been found to inhibit M^{pro} with IC₅₀ values ranging from 0.67 to 21.4 μM. One of these six compounds, Ebselen, also showed promising antiviral activity at the cellular level (FIG. 3) [Jin et al., Nature 2020, 582, 289-293].

In another study, Zhang and Yang's teams were able to highlight, by virtue of the X-ray structure, the interaction mode between M^{pro} and Carmofur (Carmofur has been used since 1980 for the treatment of colorectal cancer [Sakamoto et al., Jpn J. Clin. Oncol. 2005, 35, 536-544]). This compound establishes a covalent bond with the catalytic site, namely Cys145, while the alkyl (hydrophobic) part occupies the pocket S2 (FIG. 4) [Jin et al., Nat. Struct. Mol. Biol. 2020, 27, 529-532] (FIG. 3). The anti-SARS-COV-2 activity of Carmofur (IC₅₀=1.82 μM) has been revealed for the first time by the same team following a screening of 10,000 compounds [Jin et al., Nature, 2020, 582, 289-293].

Very recently, two new molecules called GRL-1720 and 5h have been developed by Mitsuya and colleagues as M^{pro} inhibitors. Using cell-based studies on VeroE6 cells and RNA replication by qPCR, the researchers showed that both compounds blocked SARS-COV-2 infection with EC₅₀ values of 15±4 and 4.2±0.7 μM for GRL-1720 and 5h, respectively. Further studies by X-ray diffraction analysis showed that compound 5h forms a covalent bond with M^{pro} as well as other bonds by polar interactions with several amino acid residues of the active site (FIG. 5) [Hattori et al, Nat. Commun., 2021, 12:668].

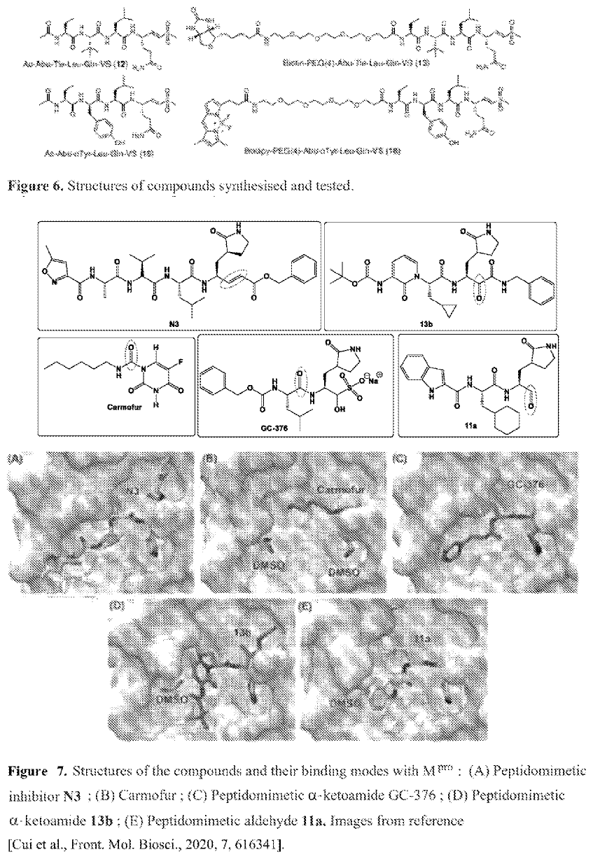

Also very recently, Rut, Drag and colleagues published the design and synthesis of pseudo-peptide inhibitors of SARS-COV-2 from a mixture of natural and non-natural amino acids (FIG. 6). The best molecule Ac-QSS-VS (15) (Ac-Abu-dTyr-Leu-Gln-VS) has an EC₅₀ of 3.7 μM. They also obtained the crystal structure by complexing another molecule called B-QS1-VS (13) (Biotin-PEG(4)-Abu-Leu-Gln-VS) with M^{pro} of SARS-COV-2. Thanks to the fluorescence of the compounds synthesised, they also monitored the interaction of the inhibitors with the active site of the SARS-COV-2 M^{pro} in nasopharyngeal epithelial cells of patients suffering from COVID-19 infection [Rut et al, Nat. Chem. Biol. 2021, 17, 222-228].

Despite recent major efforts, there is as yet no therapy to treat COVID-19. Following determination of the crystal structure of M^{pro}(Jin et al., Nature 2020, 582, 289-293), most of the studies published relate to molecular modelling, studying the interaction between the active site of M^{pro} and several bioactive compounds already on the market or in clinical development (antivirals, anticancer drugs, etc.), in order to go faster and avoid some preclinical phases (Yoshino et al., Sci. Rep. 2020, 10, 12493; Bolcato et al. Sci. Rep. 2020, 10, 20927). For example, the majority of compounds studied by molecular modelling, based on the crystal structure published by Jin et al (Jin et al., Nature, 2020, 582, 289-293), target inhibition of the M^{pro} active site by mimicking the action of the peptidomimetic N3 (FIG. 7), whose mechanism of action involves an electrophilic chemical group (a Michael acceptor) capable of covalently binding with Cys 145 (FIG. 7A). For example, in the article reported by W. Cui, K. Yang and H. Yang, the study of Carmofur (FIG. 7B) by molecular modelling shows that the carboxylic acid function of Carmofur is bound to the sulphur atom of Cys145 by a 1.8 Å covalent bond, while the fatty acid tail is inserted into the pocket S2. The entire Carmofur molecule is stabilised by numerous hydrogen bonds and hydrophobic interactions. In contrast, the inhibitor N3 (FIG. 7A) is inserted via a different mechanism, forming a covalent bond with Cys145 by Michael addition of the thiol to the vinyl group. For the binding mode between M^{pro} and the peptidomimetic a-ketoamide GC-376 and 13b (FIGS. 1C and 1D) on the one hand, and between M^{pro} and the aldehyde 11a on the other hand (FIG. 1E), modelling studies also showed that the carbonyl groups of these compounds are covalently bound to Cys145. Whereas, the other groups present on these same compounds occupy the sites S1, S2, S3 and S4 of the protease and establish hydrophobic and hydrogen interactions (Cui et al., Front. Mol. Biosci., 2020, 7, 616341).

The inventors have designed and prepared new anti-SARS-COV-2 compounds and M^{pro}(or 3CL^{Pro}) protease inhibitors that better meet practical needs, especially due to the simplicity of their preparation. These compounds could potentially be used in human therapy.

This objective is achieved by the compounds of the formulae (I) and (II) which are described below and which are the first object of the invention, these compounds being potent anti-SARS-COV-2 and M^{pro} inhibitors. Furthermore, the compounds are easy to prepare, generally in two to four steps. All the compounds of the formulae (I) and (II) are obtained very conveniently using very simple chemical reactions well known in the literature.

The present invention is directed to compounds of the following formulae (I) and (II):

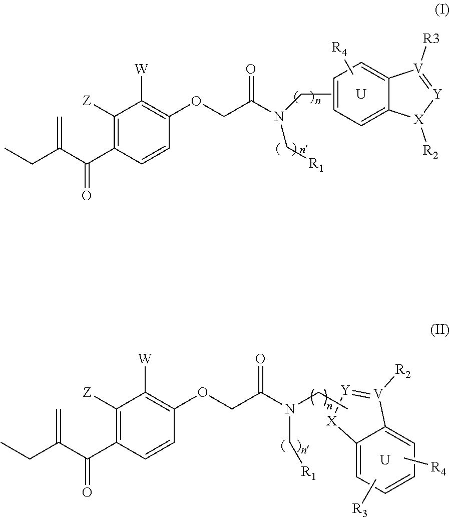

wherein:
W and Z, which may be identical or different and independently of each other, represent a hydrogen, halogen atom, hydroxyl or amine, preferably W is a hydrogen or chlorine atom and Z is a chlorine, fluorine, bromine atom or hydroxy;
V, X and Y, which may be identical or different and independently of each other, represent a carbon, nitrogen, oxygen or sulphur atom, preferably V and X are nitrogen atoms and Y is a carbon or nitrogen atom.
U is a carbon atom or one or more nitrogen atoms which replace the carbon atoms of the six-membered aromatic ring, preferably U is a carbon atom or one or two nitrogen atoms.
n and n′, which may be identical or different and independently of each other, represent the length of the alkyl, hydroxy, perfluoroalkyl, alkylthio, aminoalkyl and alkoxy chain, which may comprise from 1 to 10 carbon atoms.
R1, R2, R3 and R4, which may be identical or different and independently of each other, represent a hydrogen, halogen atom, hydroxy, alkoxy, alkyl, aryl, heteroaryl or amine, said alkyl, hydroxy, perfluoroalkyl, alkylthio, aminoalkyl and alkoxy groups may comprise from 1 to 10 carbon atoms.

For the purposes of the present invention, the term:
Alkyl: refers to a saturated, linear or branched, hydrocarbon aliphatic group having 1 to 10 carbons, preferably 1 to 2 carbon atoms. The term “branched” means that at least one lower alkyl group such as methyl or ethyl is carried by a linear alkyl chain (higher alkyl). The term “lower” alkyl refers to an alkyl having 1 or 2 carbon atoms; the term “higher” alkyl refers to a linear or branched alkyl group having from 3 to 10 carbon atoms. By way of example, alkyl groups include methyl, ethyl, n-propyl, isobutyl, tert-butyl, n-butyl and n-pentyl.
Halogen atom: designates a bromine, chlorine, iodine or fluorine atom; bromine, chlorine and fluorine being the preferred designations.
Perfluoroalkyl: designates an alkyl group as defined below in which all the hydrogen atoms have been replaced with fluorine atoms. Among perfluoroalkyl groups, trifluoromethyl and perfluoroethyl groups are preferred;
Alkoxy: designates an O-alkyl group in which the alkyl group may have the same meaning as indicated above. By way of example of an alkoxy group, mention may be made of methoxy, ethoxy, n-propoxy, iso-propoxy, n-butoxy and pentoxy groups;
Alkylthio: designates an S-alkyl group in which the alkyl group can have the same meaning as indicated above. By way of example of an alkylthio group, mention may be made of methylthio, ethylthio, propylthio, isopropylthio, butylthio and pentylamino groups;
Aminoalkyl: designates an N-alkyl group in which the alkyl group can have the same meaning as indicated above. By way of example of an aminoalkyl group, mention may be made of aminomethyl, aminoethyl, iso-propylamino, butylamino and pentylamino groups;
Aryl: designates an unsaturated, cyclic, aromatic hydrocarbon group having 4 to 6 carbons, preferably 5 to 6 carbon atoms.
Heteroaryl: designates an aryl as defined above in which one or more carbon atoms have been replaced with nitrogen, oxygen or sulphur atoms. By way of example of a heteroaryl group, mention may be made of pyridine, pyrimidine, thiophene, furan, imidazole, pyrrole and triazole groups.

According to one preferred embodiment of the invention, the compounds of the formulae (I) and (II) are selected from those in which Z and W represent halogen and alkoxy, preferably hydroxy.

According to one preferred embodiment of the invention, the compounds of the formulae (I) and (II) are selected from those in which X and Y represent a nitrogen atom and a carbon atom or two nitrogen atoms.

According to one preferred embodiment of the invention, the compounds of the formulae (I) and (II) are selected from those in which n and n′ represent a chain length of 0 to 3 carbons, preferably n and n′ are equal to 0 and 1.

According to one preferred embodiment of the invention, the compounds of the formula (I) and (II) are selected from those in which R1, R2, R3 and R4 represent hydrogen, alkyl, hydroxy and alkylamine, preferably hydrogen, methyl, aminomethyl and hydroxy.

As compounds of the formulae (I) and (II), mention may be made of: 2-(2,3-dichloro-4-(2-methylenbutanoyl)phenoxy)-N-(1-methyl-1H-indol-5-yl)acetamide of the following formula:

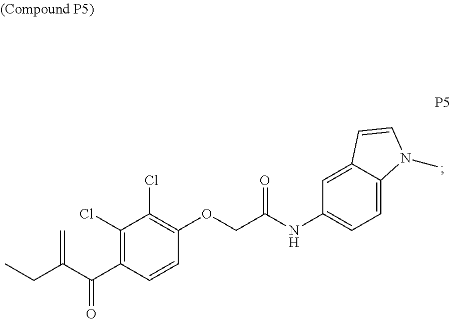 [SMILES: C=C(CC)C(=O)c1ccc(OCC(=O)Nc2ccc3c(ccn3C)c2)c(Cl)c1Cl]

2-(2,3-dichloro-4-(2-methylenebutanoyl)phenoxy)-N-(1-methyl-1H-indazol-5-yl)acetamide of the following formula:

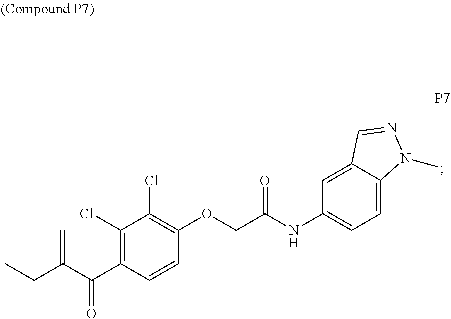 [SMILES: C=C(CC)C(=O)c1ccc(OCC(=O)Nc2ccc3c(cnn3C)c2)c(Cl)c1Cl]

2-(2,3-dichloro-4-(2-methylenebutanoyl)phenoxy)-N-(1H-indol-4-yl)acetamide of the following formula:

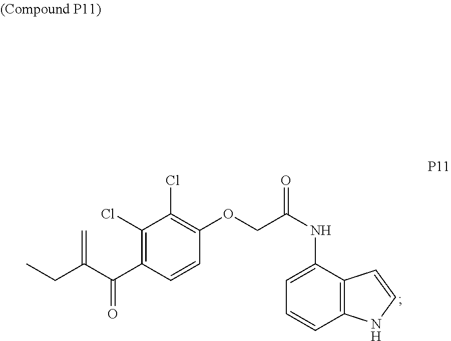 [SMILES: C=C(CC)C(=O)c1ccc(OCC(=O)Nc2cccc3[nH]ccc23)c(Cl)c1Cl]

2-(2,3-dichloro-4-(2-methylenebutanoyl)phenoxy)-N-(1H-indol-5-yl)acetamide of the following formula:

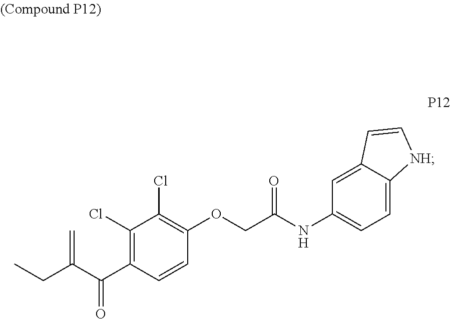 [SMILES: C=C(CC)C(=O)c1ccc(OCC(=O)Nc2ccc3[nH]ccc3c2)c(Cl)c1Cl]

2-(2,3-dichloro-4-(2-methylenebutanoyl)phenoxy)-N-(1H-indol-6-yl)acetamide of the following formula:

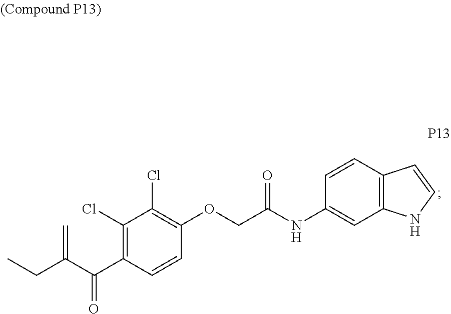 [SMILES: C=C(CC)C(=O)c1ccc(OCC(=O)Nc2ccc3cc[nH]c3c2)c(Cl)c1Cl]

2-(2,3-dichloro-4-(2-methylenebutanoyl)phenoxy)-N-(1-methyl-1H-indazol-4-yl)acetamide of the following formula:

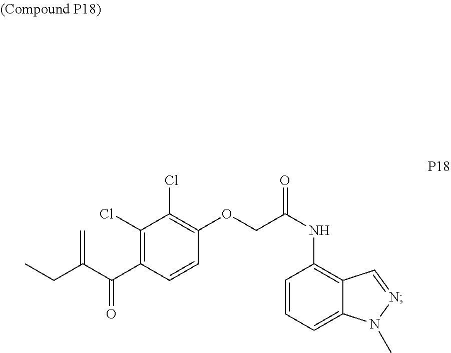 [SMILES: C=C(CC)C(=O)c1ccc(OCC(=O)Nc2cccc3c2cnn3C)c(Cl)c1Cl]

2-(2,3-dichloro-4-(2-methylenebutanoyl)phenoxy)-N-(1-methyl-1H-indazol-6-yl)acetamide of the following formula:

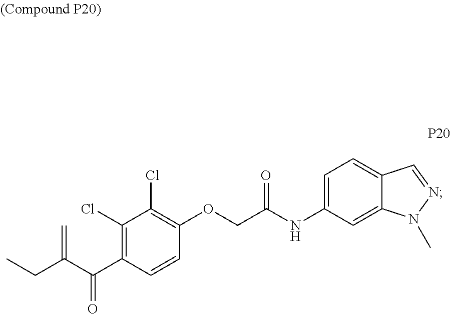 [SMILES: C=C(CC)C(=O)c1ccc(OCC(=O)Nc2ccc3cnn(C)c3c2)c(Cl)c1Cl]

2-(2,3-dichloro-4-(2-methylenebutanoyl)phenoxy)-N-(1-methyl-1H-indazol-7-yl)acetamide of the following formula:

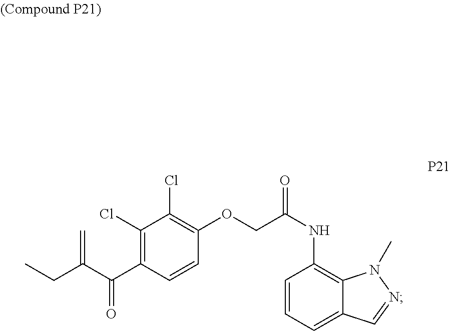 [SMILES: C=C(CC)C(=O)c1ccc(OCC(=O)Nc2cccc3cnn(C)c23)c(Cl)c1Cl]

1-(4-(2-(1H-indazol-1-yl)-2-oxoethoxy)-2,3-dichlorophenyl)-2-methylenebutan-1-one of the following formula:

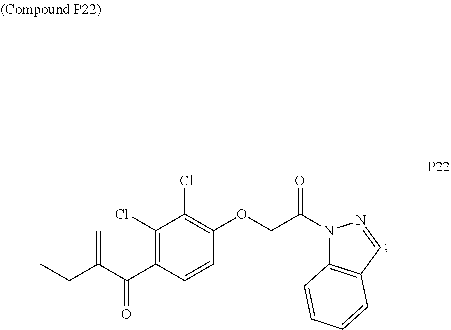 [SMILES: C=C(CC)C(=O)c1ccc(OCC(=O)n2ncc3ccccc32)c(Cl)c1Cl]

2-(2,3-dichloro-4-(2-methylenebutanoyl)phenoxy)-N-(1H-indazol-5-yl)acetamide of the following formula:

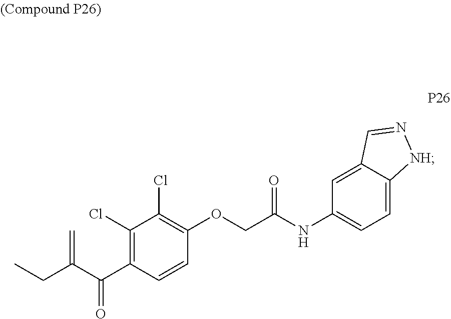 [SMILES: C=C(CC)C(=O)c1ccc(OCC(=O)Nc2ccc3[nH]ncc3c2)c(Cl)c1Cl]

2-(2,3-dichloro-4-(2-methylenebutanoyl)phenoxy)-N-(2-methyl-2H-indazol-6-yl)acetamide of the following formula:

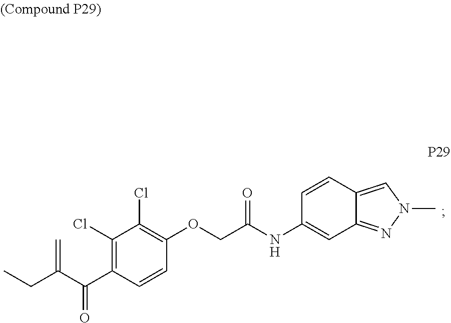 [SMILES: C=C(CC)C(=O)c1ccc(OCC(=O)Nc2ccc3cn(C)nc3c2)c(Cl)c1Cl]

2-(2,3-dichloro-4-(2-methylenebutanoyl)phenoxy)-N-(2-methyl-2H-indazol-7-yl)acetamide of the following formula:

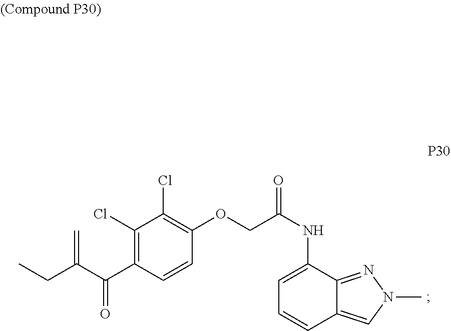 [SMILES: C=C(CC)C(=O)c1ccc(OCC(=O)Nc2cccc3cn(C)nc23)c(Cl)c1Cl]

2-(2,3-dichloro-4-(2-methylenebutanoyl)phenoxy)-N-(2-methyl-2H-indazol-4-yl)acetamide of the following formula:

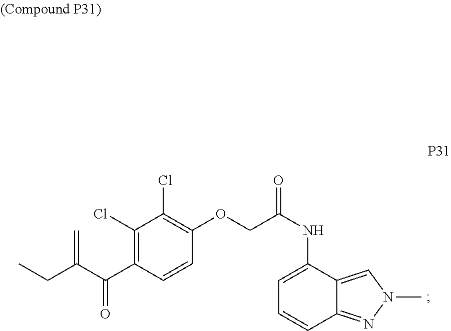 [SMILES: C=C(CC)C(=O)c1ccc(OCC(=O)Nc2cccc3nn(C)cc23)c(Cl)c1Cl]

2-(2,3-dichloro-4-(2-methylenebutanoy)phenoxy)-N-(14(4-methoxyphenyl)-sulfonyl)-1H-indazol-5-yl)acetamide of the following formula:

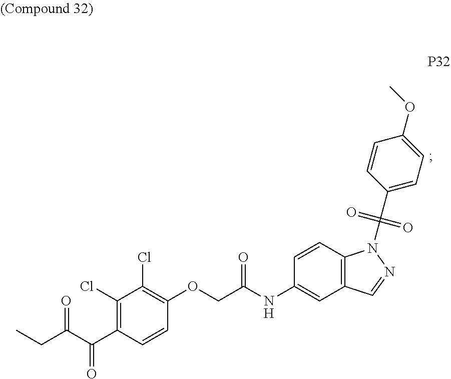

Tert-butyl 5-(2-(2,3-dichloro-4-(2-methylenebutanoyl)phenoxy)acetamido)-1H-indazole-1-carboxylate of the following formula:

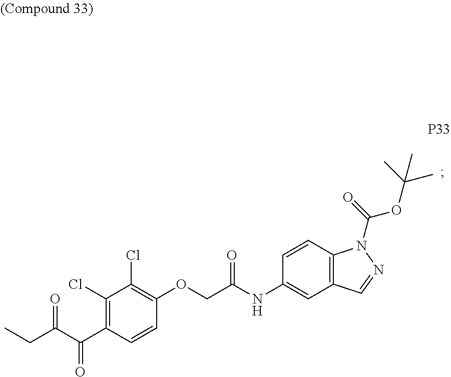 [SMILES: CCC(=O)C(=O)c1ccc(OCC(=O)Nc2ccc3c(cnn3C(=O)OC(C)(C)C)c2)c(Cl)c1Cl]

2-(2,3-dichloro-4-(2-methylenebutanoyl)phenoxy)-N-(1-(prop-2-yn-1-yl)-1H-indazol-4-yl)acetamide of the following formula:

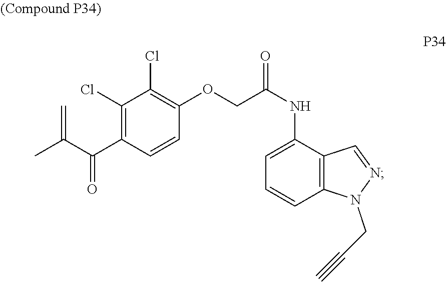 [SMILES: C#CCn1ncc2c(NC(=O)COc3ccc(C(=O)C(=C)C)c(Cl)c3Cl)cccc21]

Tert-butyl (2-(2-(2,3-dichloro-4-(2-methylenebutanoyl)phenoxy)acetamido)ethyl)-carbamate of the following formula:

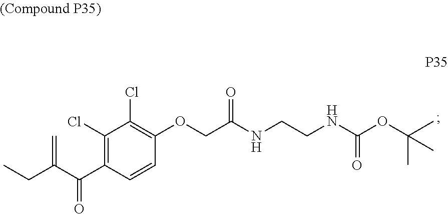 [SMILES: C=C(CC)C(=O)c1ccc(OCC(=O)NCCNC(=O)OC(C)(C)C)c(Cl)c1Cl]

Tert-butyl (1-(2-(2,3-dichloro-4-(2-methylenebutanoyl)phenoxy)acetyl)piperidin-4-yl)carbamate of the following formula:

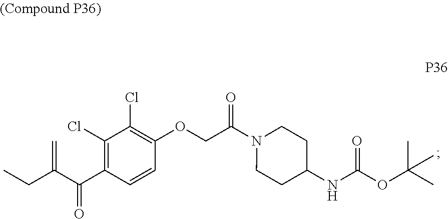 [SMILES: C=C(CC)C(=O)c1ccc(OCC(=O)N2CCC(NC(=O)OC(C)(C)C)CC2)c(Cl)c1Cl]

N-(2-chloroethyl)-2-(2,3-dichloro-4-(2-methylenebutanoyl)phenoxy)acetamide of the following formula:

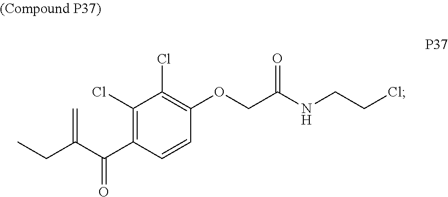 [SMILES: C=C(CC)C(=O)c1ccc(OCC(=O)NCCCl)c(Cl)c1Cl]

Tert-butyl 4-(2-(2,3-dichloro-4-(2-methylenebutanoyl)phenoxy)acetyl)piperazine-1-carboxylate of the following formula:

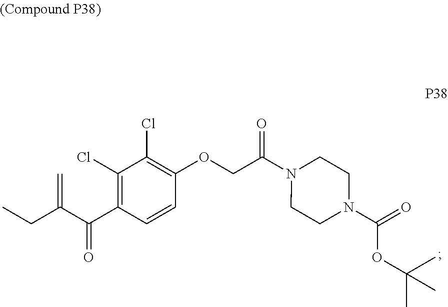 [SMILES: C=C(CC)C(=O)c1ccc(OCC(=O)N2CCN(C(=O)OC(C)(C)C)CC2)c(Cl)c1Cl]

N-butyl-2-(2,3-dichloro-4-(2-methylenebutanoyl)phenoxy)acetamide of the following formula:

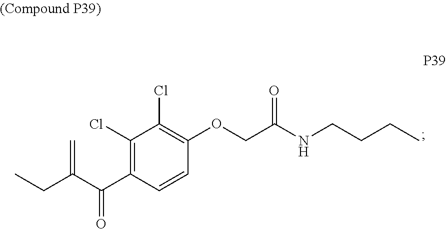 [SMILES: C=C(CC)C(=O)c1ccc(OCC(=O)NCCCC)c(Cl)c1Cl]

2-(2,3-dichloro-4-(2-methylenebutanoyl)phenoxy)-N-((1-methyl-1H-indazol-5-yl)methyl) acetamide of the following formula:

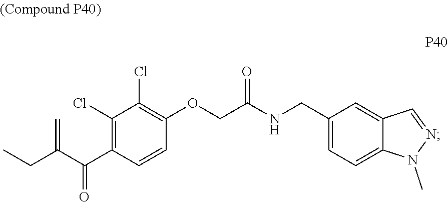 [SMILES: C=C(CC)C(=O)c1ccc(OCC(=O)NCc2ccc3c(cnn3C)c2)c(Cl)c1Cl]

1-(2,3-dichloro-4-(2-(5-methyl-1H-indazol-1-yl)-2-oxoethoxy)phenyl)-2-methylene-butan-1-one of the following formula:

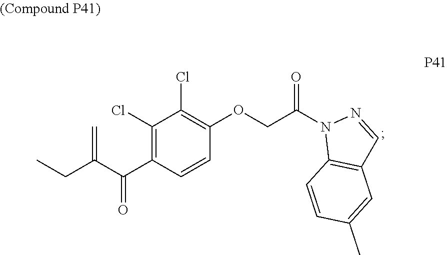 [SMILES: C=C(CC)C(=O)c1ccc(OCC(=O)n2ncc3cc(C)ccc32)c(Cl)c1Cl]

1-(2,3-dichloro-4-(2-oxo-2-(1H-pyrazolo[4,3-b]pyridin-1-yl)ethoxy)phenyl)-2-methylenebutan-1-one of the following formula:

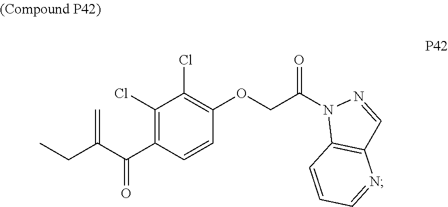 [SMILES: C=C(CC)C(=O)c1ccc(OCC(=O)n2ncc3ncccc32)c(Cl)c1Cl]

2-(2,3-dichloro-4-(2-methylenebutanoyl)phenoxy)-N-(1-methyl-1H-benzo[d]imidazol-5-yl)acetamide of the following formula:

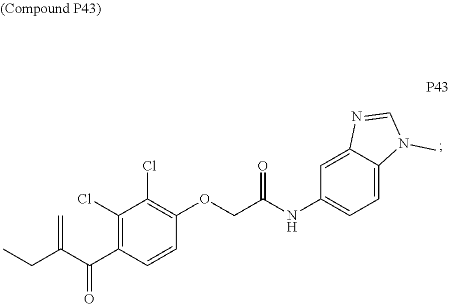 [SMILES: C=C(CC)C(=O)c1ccc(OCC(=O)Nc2ccc3c(c2)ncn3C)c(Cl)c1Cl]

### Preparation of the Compounds

All the analogues are synthesised according to the following reaction scheme 1:

Amines used are generally prepared in two steps according to the references in the literature [Swarna et al, J. Med. Chem. 2002, 45, 3, 740-743; Down et al, J. Med. Chem. 2015, 58, 18, 7381-7399; Usninn et al, Chem. Commun. 2012, 48, 2680-2682]. Ethacrynic acid (EA) is then treated with different amines via an amidation reaction carried out according to the procedure described in Scheme 1. This sequence took place in the presence of activating agents such s N,N′-dicyclohexylcarbodiimide (DCC), hydroxybenzotriazole (HOBt) and 4-dimethylaminopyridine (DMAP) in DCM at room temperature. The desired products are obtained in satisfactory yields after purification on a silica gel column.

### Synthesis Protocols and Characterisation of Different Compounds According to the Invention

Solvents have been dried according to standard methods and distilled under nitrogen before use. All the reagents have been used without prior purification from conventional commercial sources. The nuclear magnetic resonance (NMR) spectra of the ¹³C carbon and ¹H proton have been recorded on a JEOL AC500 (500 MHZ) apparatus. Chemical shifts (δ) are recorded in ppm relative to the residual TMS reference peak. JNM-ECZ500R/S1 FT NMR SYSTEM (JEOL).

### EXPERIMENTAL PROTOCOLS

### Preparation of 2-(2,3-dichloro-4-(2-methylenebutanoyl)phenoxy)-N-(1-methyl-1H-indol-5-yl)acetamide (P5)

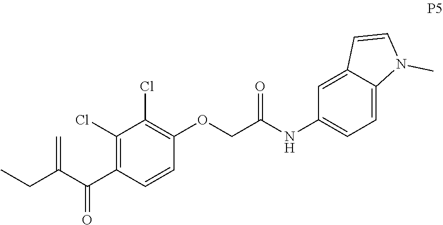 [SMILES: C=C(CC)C(=O)c1ccc(OCC(=O)Nc2ccc3c(ccn3C)c2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1-methyl-1H-indol-5-amine (24.12 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (9:1 to 8:2 (v/v)) to give the expected product P5 as a white solid (48 mg in 67% yield). 1H NMR (CDCl₃, 500 MHz), δ (ppm): 8.56 (s, 1H), 7.95 (d, J=2.0 Hz, ¹H), 7.38 (dd, J=2.0, 8.7 Hz, 1H), 7.32 (d, J=8.7 Hz, 1H), 7.25 (d, J=3.1 Hz, 1H), 7.38 (d, J=3.1 Hz, 1H), 6.97 (d, J=8.5 Hz, 1H), 6.53-6.48 (m, 1H), 5.99 (s, 1H), 5.64 (s, 1H), 4.75 (s, 2H), 3.82 (s, 3H), 2.51 (q, J=7.4 Hz, 2H), 1.18 (t, J=7.4 Hz, 3H). ¹³C NMR (CDCl₃, 126 MHZ), δ (ppm): 195.50, 164.30, 154.41, 150.27, 134.53, 134.33, 131.55, 129.90, 128.95, 128.75, 128.53, 127.32, 115.52, 112.73, 111.12, 109.44, 101.10, 68.40, 32.90, 23.40, 12.40.

### Preparation of 2-(2,3-dichloro-4-(2-methylenebutanoyl)phenoxy)-N-(1-methyl-1H-indazol-5-yl)acetamide (P7)

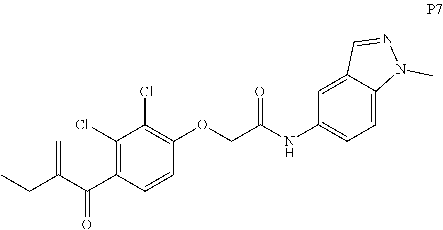 [SMILES: C=C(CC)C(=O)c1ccc(OCC(=O)Nc2ccc3c(cnn3C)c2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1-methyl-1H-indazol-5-amine (24.29 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P7 as a white solid (55 mg in 77% yield). ¹H NMR (CDCl₃, 500 MHZ), δ (ppm): 8.63 (s, 1H), 8.14 (d, J=1.5 Hz, 1H), 8.01-7.96 (m, 1H), 7.49 (dd, J=1.5, 8.9 Hz, 1H), 7.40 (d, J=8.9 Hz, 1H), 7. 24 (d, J=8.5 Hz, 1H), 6.96 (d, J=8.5 Hz, 1H), 5.99 (s, 1H), 5.63 (s, 1H), 4.74 (s, 2H), 4.09 (s, 3H), 2.50 (q, J=7.4 Hz, 2H), 1.18 (t, J=7.4 Hz, 3H). ¹³C NMR (CDCl₃, 126 MHz), δ (ppm): 195.42, 164.62, 154.33, 150.19, 137.70, 134.59, 132.85, 131.56, 129.85, 128.85, 127.36, 124.05, 123.07, 120.75, 112.05, 111.25, 109.47, 68.48, 35.63, 23.46, 12.43.

### Preparation of 2-(2.3-dichloro-4-(2-methylenebutanoyl)phenoxy)-N-(1H-indol-4-yl)acetamide (P11)

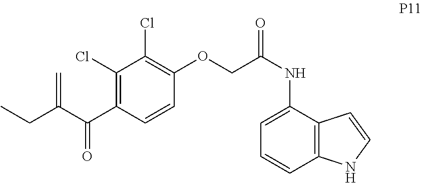 [SMILES: C=C(CC)C(=O)c1ccc(OCC(=O)Nc2cccc3[nH]ccc23)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1H-indol-4-amine (21.78 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (9:1 (v/v)) to give the expected product P11 as a white solid (37 mg in 51% yield).

### Preparation of 2-(2,3-dichloro-4-(2-methylenebutanoyl)phenoxy)-N-(1H-indol-5-yl)acetamide (P12)

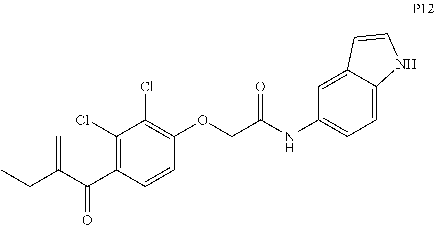 [SMILES: C=C(CC)C(=O)c1ccc(OCC(=O)Nc2ccc3[nH]ccc3c2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL) 5-aminoindole (21.81 mg, 0.165 mmol) is added at 0° ° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P12 as a white solid (44 mg in 64% yield). ¹H NMR (CDCl₃, 500 MHZ), δ (ppm): 8.57 (s, 1H), 8.36 (s, 1H), 7.97 (s, 1H), 7.41-7.29 (m, 2H), 7.27-7.18 (m, 2H), 6. 92 (d, J=8.7 Hz, 1H), 6.56 (s, 1H), 5.99 (s, 1H), 5.63 (s, 1H), 4.72 (s, 2H), 2.51 (q, J=7.4 Hz, 2H), 1.18 (t, J=7.4 Hz, 3H). ¹³C NMR (CDCl₃, 126 MHZ) δ (ppm): 195.54, 164.53, 154.44, 150.23, 134.33, 133.54, 131.55, 129.24, 128.86, 128.07, 127.27, 125.45, 123.06, 116.06, 112.66, 111.35, 111.15, 102.85, 68.46, 23.43, 12.43.

### Preparation of 2-(2,3-dichloro-4-(2-methylenebutanoyl)phenoxy)-N-(1H-indol-6-yl)acetamide (P13)

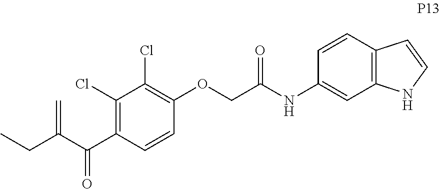 [SMILES: C=C(CC)C(=O)c1ccc(OCC(=O)Nc2ccc3cc[nH]c3c2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1H-indol-6-amine (21.78 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (9:1 (v/v)) to give the expected product P13 as a white solid (35 mg in 49% yield).

### Preparation of 2-(2.3-dichloro-4-(2-methylenebutanoyl)phenoxy)-N-(1-methyl-1H-indazol-4-yl)acetamide (P18)

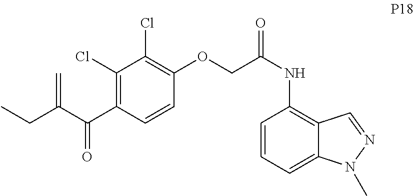 [SMILES: C=C(CC)C(=O)c1ccc(OCC(=O)Nc2cccc3c2cnn3C)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1-methyl-1H-indazol-4-amine (24.29 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P18 as a white solid (41 mg in 58% yield). ¹H NMR (CDCl3, 500 MHZ), δ (ppm): 8.96 (s, 1H), 8.07 (d, J=1.0 Hz, 1H), 7.90 (d, J=7.5 Hz, 1H), 7.40 (dd, J=8.4, 7.6 Hz, 1H), 7.26-7.19 (m, 1H), 6. 94 (d, J=8.5 Hz, 1H), 5.97 (s, 1H), 5.60 (s, 1H), 4.82-4.75 (m, 1H), 4.77 (s, 2H), 4.09 (s, 3H), 2.47 (q, J=7.4 Hz, 2H), 1.15 (t, J=7.4 Hz, 3H). ¹³C NMR (CDCl₃, 126 MHZ), δ (ppm): 195.59, 164.73, 154.14, 150.27, 140.95, 134.66, 131.82, 129.44, 129.09, 129.02, 127.48, 127.38, 122.95, 116.70, 111.13, 110.95, 106.02, 68.14, 35.92, 23.48, 12.47.

### Preparation of 2 -(2,3-dichloro-4-(2-methylenebutanoyl)phenoxy)-N-(1-methyl-1H-indazol-6-yl)acetamide (P20)

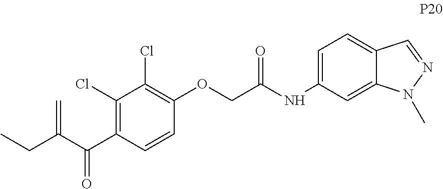 [SMILES: C=C(CC)C(=O)c1ccc(OCC(=O)Nc2ccc3cnn(C)c3c2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1-methyl-1H-indazol-6-amine (24.29 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P20 as a white solid (55 mg in 77% yield). ¹H NMR (CDCl3, 500 MHZ), δ (ppm): 8.74 (s, 1H), 8.24-8.13 (m, 1H), 7.93 (d, J=1.0 Hz, 1H), 7.68 (dd, J=8.5, 0.8 Hz, 1H), 7.23 (d, J=8.5 Hz, 1H), 6. 93 (d, J=1.6 Hz, 1H), 5.97 (s, 1H), 5.60 (s, 1H), 4.74 (s, 2H), 4.11 (s, 1H), 4.07 (s, 3H), 2.35 (q, J=7.4 Hz, 2H), 1.13 (t, J=7.4 Hz, 3H). ¹³C NMR (CDCl₃, 126 MHZ), δ (ppm): 195.40, 164.92, 156.89, 137.16, 132.80, 131.75, 129.07, 127.46, 121.86, 121.41, 119.04, 114.40, 111.28, 99.64, 68.43, 49.26, 35.78, 23.48, 12.47.

### Preparation of 2-(2.3-dichloro-4-(2-methylenebutanoyl)phenoxy)-N-(1-methy-1H-indazol-7-yl)acetamide (P21)

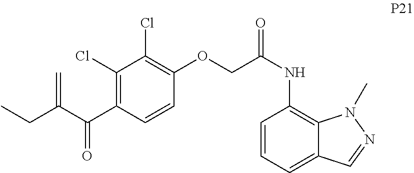 [SMILES: C=C(CC)C(=O)c1ccc(OCC(=O)Nc2cccc3cnn(C)c23)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1-methyl-1H-indazol-7-amine (24.29 mg, 0.165 mmol) is added at 0° ° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P21 as a white solid (34 mg in 44% yield). ¹H NMR (CDCl3, 500 MHZ), δ (ppm): 7.98 (s, 1H), 7.66 (dd, J=8.1, 1.0 Hz, 1H), 7.50 (dt, J=7.5, 1.0 Hz, 1H), 7.25 (d, J=8.5 Hz, 1H), 7.19-7. 13 (m, 1H), 6.97 (d, J=8.5 Hz, 1H), 5.97 (s, 1H), 5.60 (s, 1H), 4.82 (s, 2H), 4.36 (s, 1H), 4.27 (s, 3H), 2.47 (t, J=7.4 Hz, 2H), 1.15 (t, J=7.4 Hz, 3H). ¹³C NMR (CDCl₃, 126 MHz), δ (ppm): 195.52, 166.41, 156.88, 150.28, 135.04, 134.82, 133.22, 131.85, 129.07, 127.48, 126.97, 124.55, 121.13, 120.64, 118.98, 111.09, 68.41, 49.25, 38.47, 23.47, 12.47.

### Preparation of 1-(4-(2-(1H-indazol-1-yl)-2-oxoethoxy)-2.3-dichlorophenyl)-2-methylenebutan-1-one (P22)

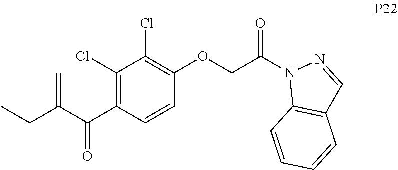 [SMILES: C=C(CC)C(=O)c1ccc(OCC(=O)n2ncc3ccccc32)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1H-indazole (19.47 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with DCM to give the expected product P22 as a white solid (41 mg in 62% yield). ¹H NMR (CDCl₃, 500 MHz), δ (ppm): 8.44 (d, J=8.4 Hz, 1H), 8.23 (s, 1H), 7.97 (d, J=8.4 Hz, 1H), 7.65-7.63 (m, 1H), 7.46-7.44 (m, 1H), 7. 15 (d, J=8.5 Hz, 1H), 6.94 (d, J=8.7 Hz, 1H), 5.96 (s, 1H), 5.71 (s, 2H), 5.65 (s, 1H), 2.49 (q, J=7.4 Hz, 2H), 1.17 (t, J=7.4 Hz, 3H). ¹³C NMR (CDCl₃, 126 MHZ) δ (ppm): 195.80, 166.41,155.46, 155.60, 150.10, 141.20, 139.00, 133.84, 131.55, 130.12, 128.50, 126.00, 125.21, 123.41, 121.20, 115.11, 111.00, 67.40, 23.41, 12.40.

### Preparation of 2-(2.3-dichloro-4-(2-methylenebutanoyl)phenoxy)-N-(1H-indazol-5-yl)acetamide (P26)

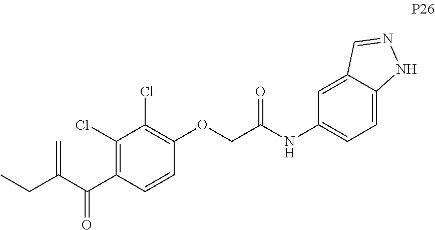 [SMILES: C=C(CC)C(=O)c1ccc(OCC(=O)Nc2ccc3[nH]ncc3c2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1H-indazol-5-amine (21.91 mg, 0.165 mmol) is added at 0° ° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P26 as a white solid (34 mg in 50% yield). RMN ¹H (CDCl₃, 500 MHZ), δ (ppm): 13.01 (s, 1H), 10.19 (s, 1H), 8.11 (s, 1H), 8.03 (s, 1H), 7.51 (d, J=8.9 Hz, 1H), 7.45 (d, J=8.9 Hz, 1H), 7. 36 (d, J=8.6 Hz, 1H), 7.20 (d, J=8.6 Hz, 1H), 6.08 (s, 1H), 5.59 (s, 1H), 4.98 (s, 2H), 2.38 (q, J=7.4 Hz, 2H), 1.08 (t, J=7.4 Hz, 3H). ¹³C NMR (CDCl₃, 126 MHz), δ (ppm): 195.60, 165.60, 156.10, 149.80, 137.05, 133.09, 132.90, 131.70, 130.00, 129.80, 128.00, 123.10, 121.60, 120.80, 112.04, 110.70, 110.60, 68.40, 23.40, 12.80.

### Preparation of 2-(2,3-dichloro-4-(2-methylenebutanoyl)phenoxy)-N-(2-methyl-2H-indazol-6-yl)acetamide (P29)

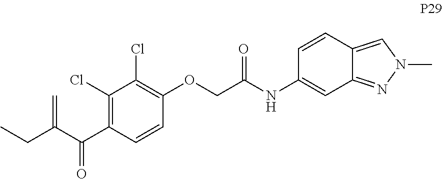 [SMILES: C=C(CC)C(=O)c1ccc(OCC(=O)Nc2ccc3cn(C)nc3c2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg g, 0.165 mmol) in DCM (5 mL), 2-methyl-2H-indazol-6-amine (24.29 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P29 as a white solid (41 mg in 58% yield). ¹H NMR (CDCl₃, 500 MHZ), δ (ppm): 8.65 (s, 1H), 7.92-7.82 (m, 1H), 7.71-7.58 (m, 1H), 7.59 (d, J=2.8 Hz, 1H), 7.35 (d, J=8.5 Hz, 2H), 7.23-7.10 (m, 1H), 6.90 (d, J=8.5 Hz, 1H), 5.95 (d, J=1.6 Hz, 1H), 5.59 (s, 1H), 5.28 (s, 1H), 4.69 (s, 2H), 4.18 (s, 3H), 2.44 (q, 7.4 Hz, 2H), 0.98 (t, J=7.4 Hz, 3H). ¹³C NMR (CDCl₃, 126 MHZ), δ (ppm): 201.31, 164.57, 150.23, 134.64, 133.00, 130.88, 129.93, 128.82, 127.97, 126.97, 125.26, 124.17, 121.09, 117.09, 115.29, 111.26, 110.90, 68.29, 48.24, 23.47, 11.06.

### Preparation of 2-(2,3-dichloro-4-(2-methylenebutanoyl)phenoxy)-N-(2-methyl-2H-indazol-7-yl)acetamide (P30)

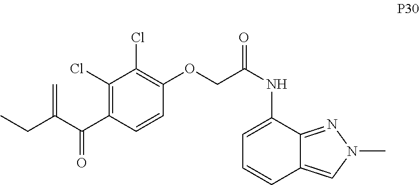 [SMILES: C=C(CC)C(=O)c1ccc(OCC(=O)Nc2cccc3cn(C)nc23)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 2-methyl-2H-indazol-7-amine (24.29 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P30 as a white solid (33 mg in 46% yield). ¹H NMR (CDCl₃, 500 MHZ), δ (ppm): 9.59 (s, 1H), 8.17 (dd, J=7.3, 0.8 Hz, 1H), 7.86 (s, 1H), 7.38 (dd, J=8.4, 0.8 Hz, 1H), 7.19 (d, J=8.4 Hz, 1H), 7.07 (dd, J=8. 5, 7.3 Hz, 1H), 6.92 (d, J=8.5 Hz, 1H), 5.95 (t, J=1.5 Hz, 1H), 5.59 (d, J=1.0 Hz, 1H), 4.75 (s, 2H), 4.20 (s, 3H), 2.47 (q, J=7.4 Hz, 2H), 1.14 (t, J=7.4 Hz, 3H). ¹³C NMR (CDCl₃, 126 MHz), δ (ppm): 195.81, 165.00, 154.65, 150.30, 141.98, 134.28, 131.60, 128.94, 127.26, 126.37, 124.25, 123.59, 122.48, 122.44, 115.82, 113.22, 111.00, 68.39, 40.57, 23.50, 12.48.

### Preparation of 2-(2.3-dichloro-4-(2-methylenebutanoyl)phenoxy)-N-(2-methyl-2H-indazol-4-yl)acetamide (P31)

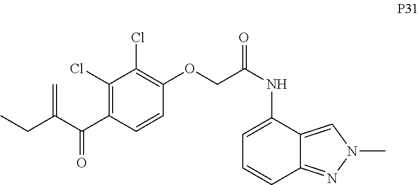 [SMILES: C=C(CC)C(=O)c1ccc(OCC(=O)Nc2cccc3nn(C)cc23)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 2-methyl-2H-indazol-4-amine (24.29 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P31 as a white solid (40 mg in 56% yield). ¹H NMR (CDCl₃, 500 MHZ), δ (ppm): 8.72 (s, 1H), 7.98 (d, J=0.9 Hz, 1H), 7.55-7.50 (m, 2H), 7.30-7.20 (m, 2H), 6.93 (d, J=8. 5 Hz, 1H), 5.97 (s, 1H), 5.59 (s, 1H), 4.74 (s, 2H), 4.22 (s, 3H), 2.47 (q, J=7.4 Hz, 2H), 1.14 (t, J=7.4 Hz, 3H). ¹³C NMR (CDCl₃, 126 MHz), δ (ppm): 195.56, 164.45, 154.20, 150.27, 149.99, 134.63, 131.70, 129.08, 128.30, 127.57, 126.33, 122.84, 121.96, 116.44, 114.78, 112.25, 111.11, 68.24, 40.70, 23.48, 12.47.

### Preparation of 2-(2.3-dichloro-4-(2-methylenebutanoyl)phenoxy)-N-(14(4-methoxyphenyl)sulfonyl)-1H-indazol-5-yl)acetamide (P32)

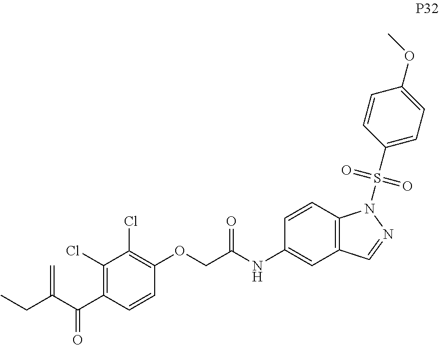 [SMILES: C=C(CC)C(=O)c1ccc(OCC(=O)Nc2ccc3c(cnn3S(=O)(=O)c3ccc(OC)cc3)c2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1((4-methoxyphenyl)sulphonyl)-1H-indazol-5-amine (50.06 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P32 as a white solid (49 mg in 51% yield). ¹H NMR (CDCl₃, 500 MHz), δ (ppm): 8.69 (s, 1H), 8.22-8.14 (m, 3H), 7.93-7.86 (m, 2H), 7.53 (dd, J=8.9, 2.1 Hz, 1H), 7.22 (d, J=8.5 Hz, 1H), 6. 93 (d, J=8.5 Hz, 1H), 6.92-6.86 (m, 2H), 5.97 (s, 1H), 5.59 (s, 1H), 4.72 (s, 2H), 3.80 (s, 3H), 2.47 (q, J=7.4 Hz, 2H), 1.14 (t, J=7.5 Hz, 3H). ¹³C NMR (CDCl₃, 126 MHz), δ (ppm): 195.55, 165.02, 164.29, 154.25, 150.25, 141.31, 137.72, 134.78, 133.18, 131.75, 129.99, 129.07, 128.79, 127.46, 126.43, 123.10, 122.74, 114.57, 113.95, 112.15, 111.33, 68.39, 55.80, 23.48, 12.47.

### Preparation of tert-butyl 5-(2-(2,3-dichloro-4-(2-methylenebutanoyl)-phenoxy)acetamido)-1H-indazole-1-carboxylate (P33)

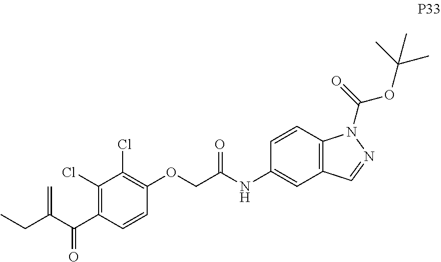 [SMILES: C=C(CC)C(=O)c1ccc(OCC(=O)Nc2ccc3c(cnn3C(=O)OC(C)(C)C)c2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL) is added at 0° C., tert-butyl 5-amino-1H-indazole-1-carboxylate (38.49 mg, 0.165 mmol). The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P33 as a white solid (49 mg in 57% yield). ¹H NMR (CDCl₃, 500 MHZ), δ (ppm): 8.70 (s, 1H), 8.28-8.24 (m, 1H), 8.19-8.13 (m, 2H), 7.52 (dd, J=9.0, 2.1 Hz, 1H), 7.22 (d, J=8.5 Hz, 1H), 6. 93 (d, J=8.5 Hz, 1H), 5.96 (t, J=1.5 Hz, 1H), 5.59 (d, J=1.0 Hz, 1H), 4.73 (s, 2H), 2.47 (q, J=7.4 Hz, 2H), 1.72 (s, 9H), 1.14 (t, J=7.4 Hz, 3H). ¹³C NMR (CDCl₃, 126 MHz), δ (ppm): 195.56, 164.93, 154.32, 150.26, 149.16, 139.61, 137.19, 134.73, 132.77, 131.74, 129.05, 127.47, 126.35, 123.11, 122.29, 115.27, 111.84, 111.31, 85.23, 68.41, 28.25, 23.47, 12.46.

### Preparation of 2-(2,3-dichloro-4-(2-methylenebutanoyl)phenoxy)-N-(1-(prop-2-yn-1-yl)-1H-indazol-4-yl)acetamide (P34)

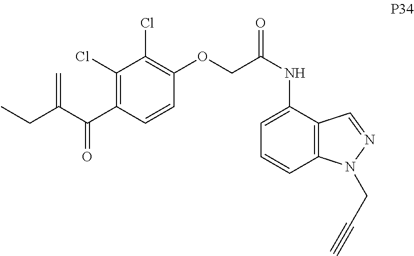 [SMILES: C#CCn1ncc2c(NC(=O)COc3ccc(C(=O)C(=C)CC)c(Cl)c3Cl)cccc21]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1-(prop-2-yn-1-yl)-1H-indazol-4-amine (28.22 mg, 0.165 mmol) is added at 0° C. The mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (9:1 (v/v)) to give the expected product P34 as a white solid (41 mg in 55% yield). ¹H NMR (CDCl₃, 500 MHZ), δ (ppm): 8.97 (s, 1H), 8.11 (dd, J=4.4, 1.0 Hz, 1H), 7.94-7.91 (m, 1H), 7.42-7.39 (m, 1H), 7.25-7.22 (m, 1H), 6.94 (d, J=8. 5 Hz, 1H), 5.96 (s, 1H), 5.60 (s, 1H), 5.19 (s, 2H), 5.06 -4.99 (m, 1H), 4.77 (s, 2H), 2.47 (q, J=7.4 Hz, 2H), 2.41 (t, J=2.6 Hz, 1H), 1.15 (t, J=7.4 Hz, 3H). ¹³C NMR (CDCl₃, 126 MHZ) δ (ppm): 195.59, 164.73, 154.12, 150.27, 140.29, 134.68, 132.55, 131.83, 130.34, 129.63, 129.05, 127.88, 127.49, 118.16, 111.78, 111.32, 110.94, 106.36, 74.02, 68.12, 39.24, 23.48, 12.47.

### Preparation of tert-butyl (2-(2-(2,3-dichloro-4-(2-methylenebutanoyl)-phenoxy)acetamido)ethyl)carbamate (P35)

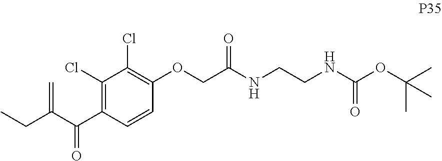 [SMILES: C=C(CC)C(=O)c1ccc(OCC(=O)NCCNC(=O)OC(C)(C)C)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), tert-butyl (2-aminoethyl)carbamate (26.44 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P35 as a white solid (48 mg in 66% yield). ¹H NMR (CDCl₃, 500 MHZ), δ (ppm): 7.20 (d, J=8.6 Hz, 1H), 6.68 (d, J=8.6 Hz, 1H), 5.97 (s, 1H), 5.60 (s, 1H), 4.88 (br, 1H), 4.59 (s, 2H), 3.55-3.51 (m, 2H), 3.36-3.32 (m, 2H), 2.49 (q, J=7.4 Hz, 2H), 1.44 (s, 9H), 1.17 (t, J=7.4 Hz, 3H). ¹³C NMR (CDCl₃126 MHz), δ (ppm): 195.52, 172.46, 167.33, 154.63, 150.22, 134.24, 131.55, 130.97, 128.78, 127.19, 123.19, 110.97, 68.26, 40.36, 39.75, 28.35, 23.45, 12.35.

### Preparation of tert-butyl (1-(2-(2,3-dichloro-4-(2-methylenebutanoyl)phenoxy)acetyl) piperidin-4-yl)carbamate (P36)

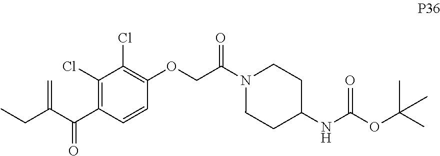 [SMILES: C=C(CC)C(=O)c1ccc(OCC(=O)N2CCC(NC(=O)OC(C)(C)C)CC2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL) tert-butyl piperidin-4-ylcarbamate (33.05 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (4:1 (v/v)) to give the expected product P36 as a white solid (39 mg in 49% yield). ¹H NMR (CDCl₃, 500 MHZ), δ (ppm): 7.16 (d, J=8.6 Hz, 1H), 6.99 (d, J=8.6 Hz, 1H), 5.96 (s, 1H), 5.62 (s, 1H), 4.93-4.73 (m, 2H), 3.47-3.42 (m, 2H), 4.04-4. 02 (m, 1H), 3.70 (br, 1H), 3.26-3.16 (m, 1H), 2.90-2.79 (m, 1H), 2.49 (q, J=7.4 Hz, 2H), 2.09-1.98 (m, 2H), 1.46 (s, 9H), 1.37-1.25 (m, 2H), 1.16 (t, J=7.4 Hz, 3H). ¹³C NMR (CDCl₃, 126 MHz), δ (ppm): 198.01, 195.81, 165.04, 155.23, 155.03, 150.26, 133.76, 128.75, 127.14, 110.64, 68.83, 44.43, 41.33, 33.95, 33.13, 32.05, 28.35, 25.63, 24.95, 23.43, 12.41.

### Preparation of N-(2-chloroethyl)-2-(2,3-dichloro-4-(2-methylenebutanoyl)phenoxy)acetamide (P37)

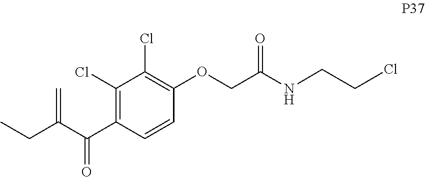 [SMILES: C=C(CC)C(=O)c1ccc(OCC(=O)NCCCl)c(Cl)c1Cl]

To a solution of DCC (37.45 mg, 0.182 mmol), HOBt (30.32 mg, 0.198 mmol), DMAP (2.02 mg, 0.017 mmol), triethylamine (0.033 mL, 0.248 mmol) and EA (50 mg, 0.165 mmol) in DCM (5 mL), 2-chloroethylamine hydrochloride (19.14 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with DCM to give the expected product P37 as a white solid (33 mg in 55% yield). ¹H NMR (CDCl₃, 500 MHz), δ (ppm): 7.24 (br, 1H), 7.22 (d, J=8.6 Hz, 1H), 6.89 (d, J=8.6 Hz, 1H), 5. 98 (s, 1H), 5.61 (s, 1H), 4.62 (s, 2H), 3.84-3.66 (m, 4H), 2.50 (q, J=7.4 Hz, 2H), 1.17 (t, J=7.4 Hz, 3H). ¹³C NMR (CDCl₃126 MHz), δ (ppm): 195.50, 166.09, 154.40, 150.20, 134.30, 131.50, 128.70, 127.02, 110.09, 110.08, 68.10, 43.05, 40.70, 23.40, 12.40.

### Preparation of tert-butyl 4-(2-(2,3-dichloro-4-(2-methylenebutanoyl)phenoxy)acetyl)piperazine-1-carboxylate (P38)

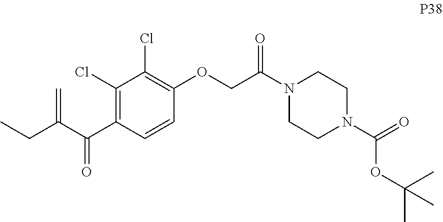 [SMILES: C=C(CC)C(=O)c1ccc(OCC(=O)N2CCN(C(=O)OC(C)(C)C)CC2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), tert-butyl piperazine-1-carboxylate (30.69 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (9:1 to 8:2 (v/v)) to give the expected product P38 as a white solid (43 mg in 56% yield). ¹H NMR (CDCl₃, 500 MHz), δ (ppm): 7.17 (d, J=8.5 Hz, 1H), 6.99 (d, J=8.5 Hz, 1H), 5.96 (s, 1H), 5.61 (s, 1H), 4. 85 (s, 2H), 3.62-3.55 (m, 4H), 3.47-3.45 (m, 4H), 2.55-2.41 (m, 2H), 1.49 (s, 9H), 1.16 (t, J=7.4 Hz, 3H). ¹³C NMR (CDCl₃, 126 MHZ), δ (ppm): 195.77, 165.40, 155.08, 154.42, 150.18, 133.85, 131.48, 128.72, 127.10, 110.64, 80.52, 68.77, 45.48, 42.13, 33.97, 29.70, 28.37, 23.42, 12.40, 12.39.

### Preparation of N-butyl-2-(2.3-dichloro-4-(2methylenebutanoyl)phenoxy)acetamide (P39)

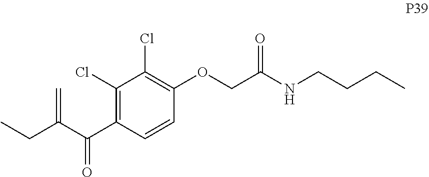 [SMILES: C=C(CC)C(=O)c1ccc(OCC(=O)NCCCC)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), n-butylamine (12.05 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (9:1 (v/v)) to give the expected product P39 as a white solid (39 mg in 65% yield). ¹H NMR (CDCl₃, 500 MHZ), δ (ppm): 7.19 (d, J=8.6 Hz, 1H), 6.87 (d, J=8.6 Hz, 1H), 6.79 (br, 1H), 5.96 (s, 1H), 5.59 (s, 1H), 4.57 (s, 2H), 3. 39 (q, J=7.4 Hz, 2H), 2.47 (q, J=7.4 Hz, 2H), 1.63-1.53 (m, 2H), 1.47-1.32 (m, 2H), 1.15 (t, J=7.4 Hz, 3H), 0.95 (t, J=7.4 Hz, 3H). ¹³C NMR (CDCl₃, 126 MHz), δ (ppm): 195.50, 166.51, 154.50, 150.10, 134.10, 131.40, 128.70, 127.20, 122.80, 110.80, 68.20, 38.80, 31.40, 23.40, 19.90, 13.70, 12.30.

### Preparation of 2-(2,3-dichloro-4-(2-methylenebutanoyl)phenoxy)-N-((1-methyl-1H-indazol-5-yl)methyl)acetamide (P40)

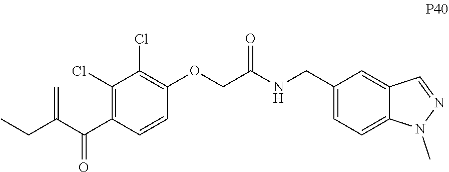 [SMILES: C=C(CC)C(=O)c1ccc(OCC(=O)NCc2ccc3c(cnn3C)c2)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), (1-methyl-1H-indazol-5-yl)methanamine (26.56 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (9:1 (v/v)) to give the expected product P40 as a white solid (37 mg in 51% yield). ¹H NMR (CDCl₃, 500 MHZ), δ (ppm): 7.94 (d, J=1.0 Hz, 1H), 7.69 (dd, J=8.3, 0.8 Hz, 1H), 7.33 (t, J=1.0 Hz, 1H), 7.17 (d, J=8.5 Hz, 1H), 7.08 (dd, J=8.3, 1.4 Hz, 1H), 6.86 (d, J=8. 5 Hz, 1H), 5.93 (s, 1H), 5.55 (s, 1H), 4.71 (d, J=6.0 Hz, 2H), 4.64 (s, 2H), 4.23 (br, 1H), 4.05 (s, 3H), 2.45 (q, J=7.4 Hz, 2H), 1.12 (t, J=7.4 Hz, 3H). RMN ¹³C (CDCl₃, 126 MHZ) δ (ppm): 195.64, 166.87, 156.96, 154.51, 150.25, 140.14, 136.07, 134.39, 132.77, 131.61, 128.96, 127.34, 123.62, 121.77, 120.59, 111.05, 107.86, 68.36, 43.60, 35.69, 23.47, 12.46.

### Preparation of 1-(2,3-dichloro-4-(2-(5-methyl-1H-indazol-1-yl)-2-oxoethoxy)phenyl)-2-methylenebutan-1-one (P41)

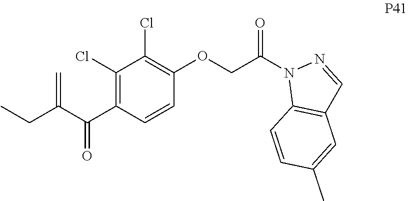 [SMILES: C=C(CC)C(=O)c1ccc(OCC(=O)n2ncc3cc(C)ccc32)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 5-methyl-1H-indazole (21.78 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with (DCM to give the expected product P41 as a white solid (40 mg in 58% yield). ¹H NMR (CDCl₃, 500 MHz), δ (ppm): 8.26 (d, J=8.5 Hz, 1H), 8.11 (s, 1H), 7.55 -7.49 (m, 1H), 7.41 (dd, J=8.5, 1.6 Hz, 1H), 7.11 (d, J=8. 5 Hz, 1H), 6.89 (d, J=8.5 Hz, 1H), 5.93 (s, 1H), 5.66 (s, 2H), 5.62 (s, 1H), 2.48 (s, 3H), 2.47 -2.42 (m, 2H), 1.13 (t, J=7.4 Hz, 3H). ¹³C NMR (CDCl₃, 126 MHZ) δ (ppm): 196.07, 166.34, 155.78, 150.25, 141.09, 137.55, 135.34, 133.83, 131.93, 129.17, 128.80, 126.92, 126.58, 123.47, 120.74, 114.79, 111.02, 67.45, 23.51, 21.42, 12.47.

### Preparation of 1-(2,3-dichloro-4-(2-oxo-2-(1H-pyrazolo[4,3-b]pyridin-1-yl)ethoxy)phenyl)-2-methylenebutan-1-one (P42)

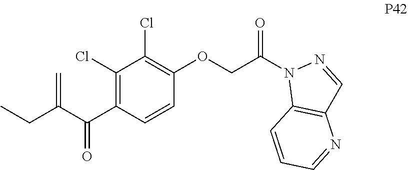 [SMILES: C=C(CC)C(=O)c1ccc(OCC(=O)n2ncc3ncccc32)c(Cl)c1Cl]

To a solution of DCC (37.45 mg, 0.182 mmol), HOBt (30.32 mg, 0.198 mmol), DMAP (2.02 mg, 0.017 mmol) and EA (50 mg, 0.165 mmol) in DCM (5 mL), 1H-pyrazolo[4,3-b]pyridine (19.64 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (9:1 (v/v)) to give the expected product P42 as a white solid (35 mg in 53% yield). ¹H NMR (CDCl₃, 500 MHz), δ (ppm): 8.77 (dd, J=4.6, 1.4 Hz, 1H), 8.67 (dt, J=8.4, 1.4 Hz, 1H), 8.43 (d, J=0.9 Hz, 1H), 7.52 (dd, J=8.4, 4.6 Hz, 1H), 7. 13 (d, J=8.4 Hz, 1H), 6.91 (d, J=8.4 Hz, 1H), 5.94 (t, J=1.5 Hz, 1H), 5.70 (s, 2H), 5.61 (s, 1H), 2.46 (q, J=7.4, 2H), 1.13 (t, J=7.4 Hz, 3H). ¹³C NMR (CDCl₃, 126 MHz): 195.97, 166.81, 155.54, 150.25, 149.26, 144.20, 141.89, 134.14, 132.72, 131.74, 128.86, 126.89, 124.03, 123.62, 123.01, 111.08, 67.27, 23.49, 12.47.

### Preparation of 2 -(2,3 -dichloro-4-(2-methylenebutanoyl)phenoxy)-N-(1-methyl-1H-benzo[d]imidazol-5-yl)acetamide (P43)

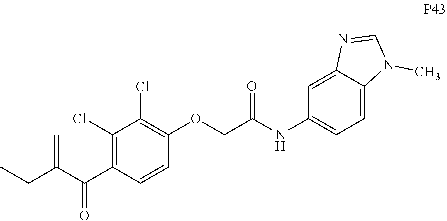 [SMILES: C=C(CC)C(=O)c1ccc(OCC(=O)Nc2ccc3c(c2)ncn3C)c(Cl)c1Cl]

To a solution of DCC (37.45 mg, 0.182 mmol), HOBt (30.32 mg, 0.198 mmol) DMAP (2.02 mg, 0.017 mmol) and EA (50 mg, 0.165 mmol) in DCM (5 mL), 1-methyl-1H-benzo[d]imidazol-5-amine (24.26 mg, 0.165 mmol) is added at 0° C. The reaction mixture is stirred overnight at room temperature. After extraction with ethyl acetate, the combined organic phases are washed with a saturated sodium chloride solution, dried over magnesium sulphate, filtered and then concentrated under pressure. The residue obtained is purified by column chromatography eluting with a mixture (DCM/EtOAc (9:1 (v/v)) to give the expected product P43 as a white solid (56 mg in 79% yield). ¹H NMR (CDCl₃, 500 MHz), δ (ppm): 8.66 (s, 1H), 8.05 (d, J=1.8 Hz, 1H), 7.98 (s, 1H), 7.63 (dd, J=1.8, 8.6 Hz, 1H), 7.40 (d, J=8.6 Hz, 1H), 7. 25 (d, J=8.5 Hz, 1H), 6.97 (d, J=8.5 Hz, 1H), 5.99 (s, 1H), 5.63 (s, 1H), 4.76 (s, 2H), 3.88 (s, 3H), 2.51 (q, J=7.4 Hz, 2H), 1.18 (t, J=7.4 Hz, 3H). ¹³C NMR (CDCl₃, 126 MHZ): 195.41, 164.52, 154.32, 150.10, 144.51, 143.91, 134.51, 132.20, 131.70, 131.61, 128.82, 127.31, 123.10, 116.81, 112.10, 111.21, 109.50, 68.41, 31.11, 23.41, 12.42.

### BIOLOGICAL ACTIVITIES

### Protocols

The quickest way to find effective drugs against COVID-19 is to conduct clinical trials with drugs already used against other diseases, since we know how to administer them and we know the effective doses thereof (drug repositioning strategy). However, inhibitors of a given protease are not necessarily effective in blocking another protease, which has different structural and functional properties. Molecules from the family of a, B-unsaturated ketones (Michael acceptors), capable of blocking 3CLpro protease of the SARS-COV-2, represent a real hope for treating patients. Some have already been used clinically, such as Telaprevir and Boceprevir, which were among the first protease inhibitors prescribed in 2011 to treat hepatitis C patients. The development of protease inhibitors should benefit from substantial resources to rapidly have an effective treatment against COVID-19.

We used a microneutralisation test, described by Amanat F et al [Amanat et al, Curr Protoc Microbiol 2020, 58, e108], to quantitatively evaluate whether antibodies or drugs can block entry and/or replication of SARS-COV-2 in vitro. To do this, microneutralisation tests are performed in a 96-well format, which allows a medium throughput. While the test initially described by Amanat et al. [Amanat et al., Curr Protoc Microbiol 2020, 58, e108] is based on staining of the virus nucleoprotein (NP) by Elisa test, we opted for a test based on RT-PCR by amplification of the genes coding respectively for the virus nucleoprotein (N), the RNA-dependent polymerase (R) and the envelope gene (E). This approach, also used by David et al [David et al, Nature 2020, 583, 459-468], enables the % inhibition to be quantitatively evaluated, thus corroborating visual observations of the cytopathic effect (CPE).

### EXPERIMENTAL PART

### Biology

The anti-SRAS-COV-2 activity of the candidate molecules has been studied in vitro in Vero cells. The characteristics of the infection of Vero cells by SARS-COV-2 are described by Yajing [Yao et al, Virol Sin. 2020, 35, 348-350]. The Vero cell line has been derived from green monkey kidney cells isolated in 1962. The Vero-E6 and Vero cells used in this study have been obtained from two cell banks (INRA Toulouse, France) and (Société Biopharma, Rabat, Morocco) at passages 39 and 159 respectively. A culture medium formulation with glucose and L-glutamine has been used.

Culture of Vero-E6 and Vero cells

Cell cultures and infections with the SARS-COV-2 virus have been performed in a biosafety level 3 (BSL-3) laboratory. Cells have been cultured in culture medium with 6% fetal calf serum (FCS) and 2% donor serum (DS). This will hereafter be referred to as reference medium (RM). The first step of cell propagation is performed in 175 cm2 T static culture flasks seeded with 30,000 cell.cm⁻² for Vero-E6 cells and 40,000 cell.cm-2 for Vero cells. These cultures are placed in a controlled incubator at 37° C. and 5% CO₂. For cell passages, the cells have been trypsinised and the cell suspension collected and used for subsequent seeding. The culture volume has then been adjusted with fresh ECM, which had been previously brought to 37° C.

### Isolation and Confirmation of SARS-COV-2

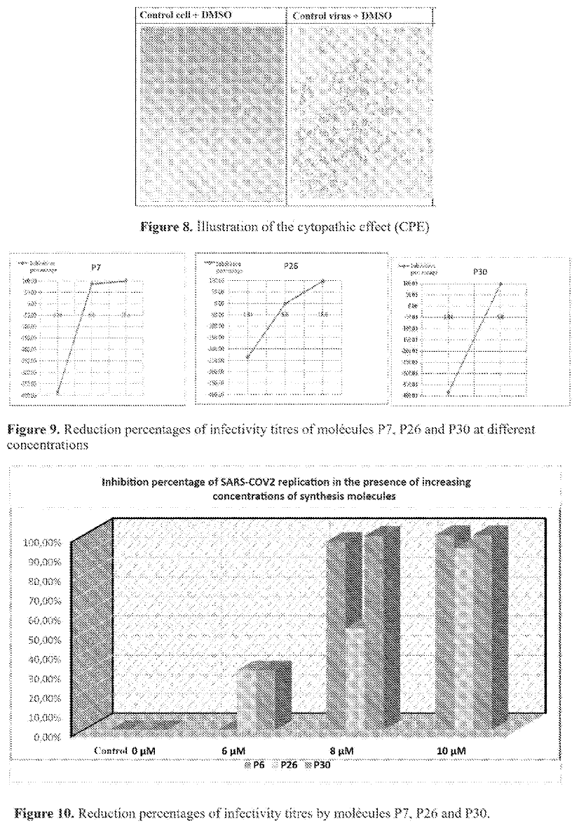

Isolation of the SARS-COV-2 virus has been performed in the Biosafety Laboratory L3 (BSL-3) from two samples (nasopharyngeal and oropharyngeal found positive in a female patient (279 CC) hospitalised at the Centre de Virologie et Maladies Infectieuses Tropicales (CVMIT), Hopital Militaire d′Instruction Mohammed V, Rabat. Viral isolation is performed according to the protocol described by (Harcourt et al., bioRxiv, 2020) using a maintenance medium called INOC. The cytopathic effect (CPE) is visible from the first passage (FIG. 8). A confirmation test for virus propagation in vero cells has been performed on the cell supernatant extracted using the viral RNA mini kit (QIAGEN, Hilden, Germany) and amplified by qRT-PCR using the IVD GeneFinder™ COVID-19 PLUS RealAmp assay kit (Korea).

### Whole Genome Sequencing of the Isolated SARS-COV-2 Virus in Culture

Sequencing of the complete viral genome has been carried out using NGS (lon proton, ThermoFisher) and Sanger sequencing. The viral genetic material used for sequencing is extracted from the passage P4 of strain 279 CC on Vero-E6. The sequence of the complete genome obtained by the Sanger technique has been deposited in the international GISAID database under the reference hCoV-19/Morocco/HMIMV-279CC/2020 Accession ID: EPI-ISL-971451.

### Virus Adaptation, Propagation, Production and Harvesting

The 279 CC virus isolated from Vero-E6 on INOC medium is then adapted on Vero cells from passage 2 (P2). Virus propagation and production are carried out in T500 cm² static culture flasks with INOC medium. Viral production lasts 5 days post-infection. Daily aliquoting allow monitoring the increase in virus titre and monitoring cell growth and metabolism. The virus is harvested on the 5th day after infection. The culture supernatant is then clarified by centrifugation, aliquoted and frozen at -80° C.

### Determination of Virus Titre

Infectious titres have been determined in the culture supernatants of infected cells and calculated according to the Reed-Muench technique (1938). They have been obtained using the limiting dilution infection technique and are expressed as TCID₅₀/mL (50% Tissue Culture Infectious Dose) or log TCID₅₀/mL.

By way of example of compounds of the formulae (1) or (2), compounds P7, P26 and P30 have been selected to illustrate the biological studies carried out.

### Screening of Synthesised Molecules to Evaluate Their Antiviral Effects

### Determination of Molecule Concentrations

The three compounds are solubilised in DMSO according to their Molarity.

### Cytotoxicity Study

The cytotoxicity study mainly consists in evaluating cell viability via a fluorescent dye, propidium iodide (logos, Biosystems, USA). It is carried out after 24 and 48 h of incubation using an automatic cell counter integrated with fluorescence optics and image analysis software (Luma, logos, Biosystems, USA).

The cytotoxicity of the different compounds is determined by the mean cell count for each test in a plate of uninfected cells. The results are set forth in FIG. 1A and are expressed in percent (%).

### Determination of the Antiviral Effect Of Molecules

Molecule antiviral screening cultures are carried out in 96-well culture plates with a volume of MEC medium of 100 μl at a density of 2.5×10⁵ cells/mL. The protocol used follows a prophylactic approach (4h incubation before in vitro infection). Indeed, cells are incubated in the presence or absence of the compounds tested for 4 h and then infected at an MOI (Multiplicity of Infection) of 0.04 for a duration of 48 h in INOC at 37° C. under 5% CO₂.

The effect on viral production (antiviral effect) in vitro is measured by qRT-PCR and by determining infectivity titres (logDITC₅₀/mL or DITC₅₀/mL) in Vero cells after 48 h of incubation. The infectivity titre ratio in each condition is expressed as a function of the infectivity titre measured in the control condition (no treatment).

The infectivity titres measured in the experimental conditions in the presence of the compounds tested are greatly reduced compared with the control condition, where the cells have been infected but not treated.

Indeed, the molecules P7 (16.6 μM), P26 (16.6 μM) and P30 (5.54 μM) allow reduction in the infectivity titres by 100%, 97.96% and 96.75% respectively compared with the control (see FIG. 9).

Based on these results, a wider range of concentrations has been tested for each of the three compounds on Vero cells under the same experimental conditions, in order to determine the effective concentration range, and thus determine an IC₅₀ (half maximal Inhibitory Concentration), that is the dose required to obtain 50% inhibition of viral production (see FIG. 10).

Indeed, the molecules P7, P26 and P30 at 10 μM allow reduction in infectivity titres by 100%, 93.09% and 100% respectively compared with the control (see FIG. 2). At 8 μM, the inhibition percentage of these molecules is in the order of 96.70%, 52.14% and 99.67% respectively. At 6 μM, only P26 and P30 inhibited viral replication by around 30%, while P7 did not inhibit virus replication at all at this concentration.

The inhibitory effect of the 3 molecules on replication of the SARS-COV-2 strain has been confirmed by RT-PCR on three virus genes, namely the gene RdRP, the gene N and the gene E (Table 1).

The IC₅₀ values for these three compounds are listed in the following table (Table 2):

These IC₅₀ values are relatively low compared with the usual concentrations known for these compounds in their non-infectious applications. These values are also a long way from the cytotoxicity concentrations (CC₅₀).

### Structure of the SARS-COV-2 M^{pro} Protein

The structure of 3CLpro (M^{pro}) has been downloaded from the Protein Data Bank (PDB ID: 6LU7) this structure is a complex of SARS-COV-2 3CL^{pro} with its inhibitor covalently bound to Cys145. [Jin et al, Nature 2020, 582, 289-293]. The structure has been energetically minimised in Rosetta [Leaver-Fay et al, Methods Enzymol. 2011, 487, 545-574]

### Docking Procedure

1-Optimisation of the structure of 3CL^{pro} (M^{pro}) (PDB ID: 6LU7) by the addition of its polar hydrogens as well as the partial charges of all its atoms. The protein backbone has been fixed during minimisation. The lowest score model has been selected from among 1000 models. MGLTools (version 1.5.6) has been used to generate the PDBQT file for docking.

By way of example of compounds of the formulae (1) or (2) compound P7 has been selected to illustrate the molecular modelling studies.

### 2-Ligand Optimisation

The ligands selected for molecular docking have been optimised by adding partial charges and hydrogen atoms followed by energy minimisation using the PRODRG server (http://davapcl.bioch.dundee.ac.uk/cgi-bin/prodrg/).

### 3-Preparation of the Configuration File and Potential Grid Calculation

The configuration file has been prepared to run AutoDock Vina. AutoDockTools is used to prepare the “input.PDBQT” file for M^{pro} and to define position and dimensions of the box (X; Y; Z). The size of the grid has been set to 20×20×20 points (x, y and z), and the centre of the grid to x, y and z dimensions of −10.729204, 12.417653 and 68.816122, respectively. The preparation file has been saved in “.PDBQT” format.

### 4-Molecular Docking

The molecular docking process is used to search for possible ligand positions and orientations and to make the most of the specificity of the docking site and the interaction potential of the docked ligand. AutoDock Vina (version 1.1.2) [Trott et al, J. Comput. Chem. 2010, 31, 455-461; Zhang et al, J. Mol. Recognit. 2016, 29, 520-527] has been used to dock the synthesised molecules at the substrate binding pocket of M^{pro}(PDB ID: 6LU7). The ligand docking simulation has been kept flexible while the receptor, made rigid.

### 5-Search for Docking Solutions

The best docking solution is to look for the most probable positions and orientations in relation to the active pocket with the lowest energy in kcal.mol-1, taking account of the different ways in which each ligand binds to the receptor (Table 3).

### 5-Analysis of Results

The best solutions or poses have been then evaluated by Discovery Studio Biovia 2021 software (Dassault Systèmes, San Diego, California, USA) and viewed using PyMOL by docking score, ranking and distance between the reactive atom and the sulphur atom of Cys145 in the original structure as described previously [Ai et al., J. Chem. Inf. Model. 2016, 56, 1563-1575].

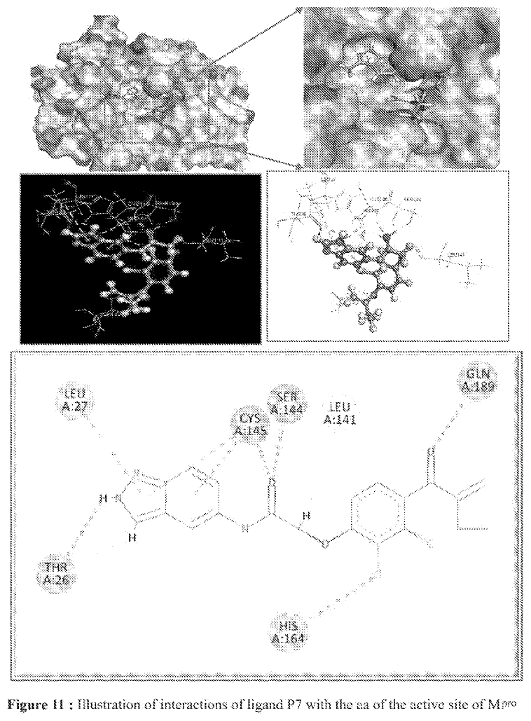

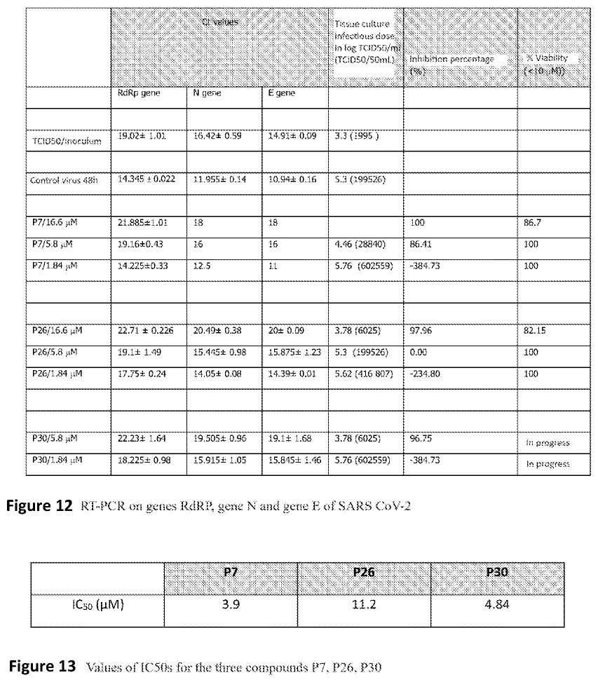

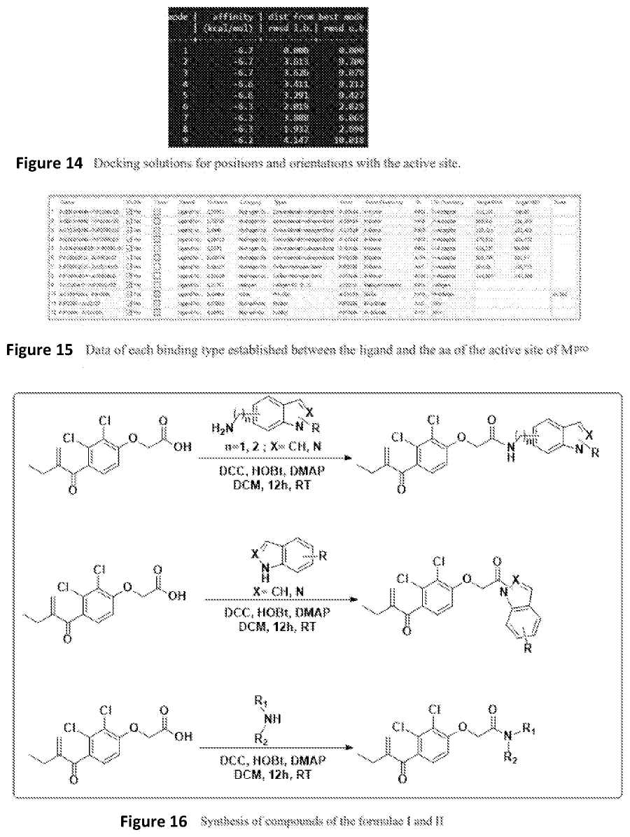

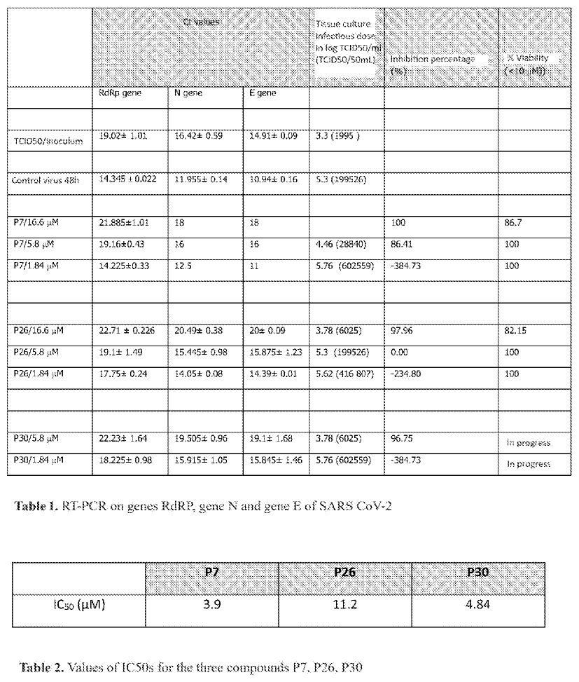

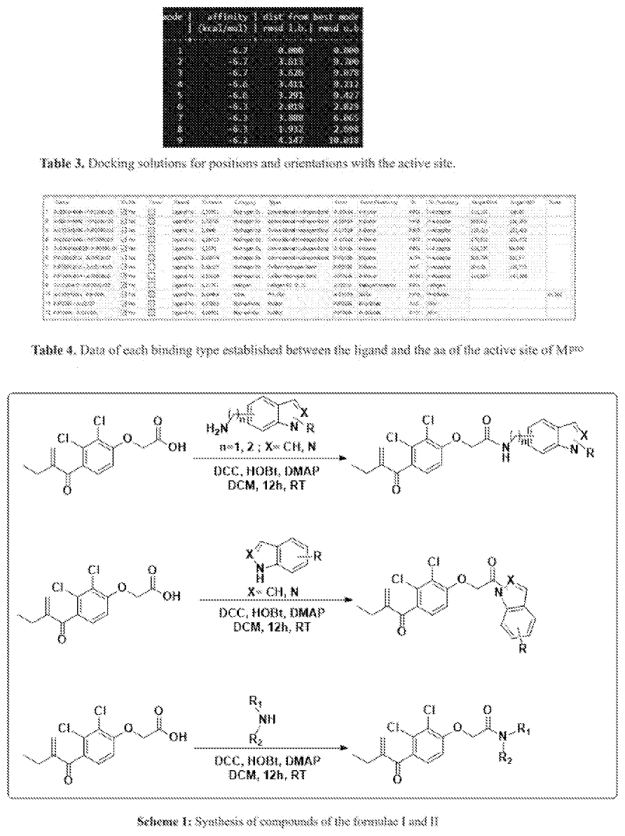

Post-docking analyses showed sizes and locations of the binding sites, hydrogen bond interactions, hydrophobic interactions and bond distances as interaction radii of <5 Å from the anchored ligand position. The compounds have been docked to the active site, then the binding poses of each ligand have been observed and their interactions with the protein have been characterised, and the most energetically favourable conformations of each ligand have been selected (FIG. 11).

The physicochemical properties of the synthetic molecules (Table 4) met the criteria of Lipinski's rule of five, also known as Lipinski's druglikeness rule. These rules enable evaluation of the structural similarities of compounds with those of active oral drugs and are based on physicochemical profiles. Molecular weights and hydrogen bonding interactions between donors and acceptors are crucial structural determinants of protein targets and ligand binding sites [Lipinski et al, Adv. Drug Deliv. Rev. 2016, 101, 34-41; Lipinski et al, Adv. Drug Deliv. Rev. 2001, 46, 3-26; Zhang et al, Curr. Opin. Biotechnol. 2007, 18, 478-88]. In particular, compounds are more likely to be permeable and active as ligands when they have no more than 5 hydrogen bond donors and no more than 10 hydrogen bond acceptors, a molecular mass of less than 500 and calculated log P values (CLog P) of less than 5 [Lipinski et al., Adv. Drug Deliv. Rev. 2001, 46, 3-26; Zhang et al, Curr. Opin. Biotechnol. 2007, 18, 478-88].

1. A compound having one of the following formulae (I) and (II): 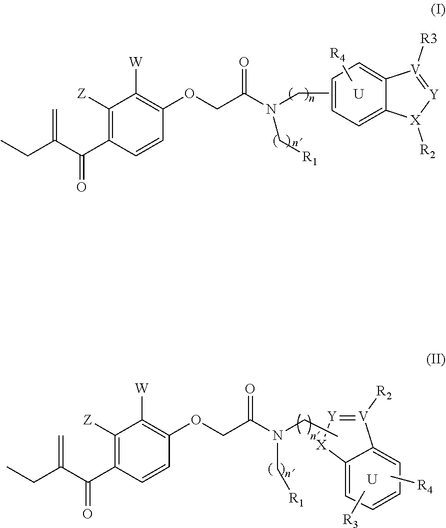
wherein:
W and Z, which may be identical or different and independently of each other, represent a hydrogen, halogen atom, hydroxyl or amine;
V, X and Y, which may be identical or different and independently of each other, represent a carbon, nitrogen, oxygen or sulphur atom;
U is a carbon atom or one or more nitrogen atoms which replace the carbon atoms of the six-membered aromatic ring, preferably U is a carbon atom or one or two nitrogen atoms;
n and n′, which may be identical or different and independently of each other, represent the length of the alkyl, hydroxy, perfluoroalkyl, alkylthio, aminoalkyl and alkoxy chain, which may comprise from 1 to 10 carbon atoms,
R1, R2, R3 and R4, which may be identical or different and independently of each other, represent a hydrogen, halogen atom, hydroxy, alkoxy, alkyl, aryl, heteroaryl or amine, said alkyl, hydroxy, perfluoroalkyl, alkylthio, aminoalkyl and alkoxy groups may comprise from 1 to 10 carbon atoms.

2-The compound according to claim 1, wherein the formulae (I) or (II) are 2-(2,3-dichloro-4-(2-methylenbutanoyl)phenoxy)-N-(1-methyl-1H-indol-5-yl)acetamide of the following formula: 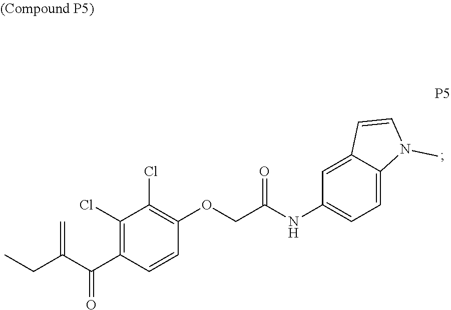 [SMILES: C=C(CC)C(=O)c1ccc(OCC(=O)Nc2ccc3c(ccn3C)c2)c(Cl)c1Cl]
2-(2,3-dichloro-4-(2-methylenebutanoyl)phenoxy)-N-(1-methyl-1H-indazol-5-yl)acetamide of the following formula: 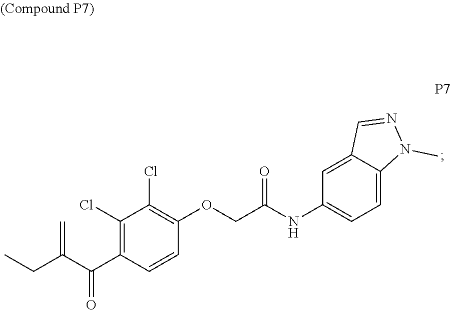 [SMILES: C=C(CC)C(=O)c1ccc(OCC(=O)Nc2ccc3c(cnn3C)c2)c(Cl)c1Cl]
2-(2,3-dichloro-4-(2-methylenebutanoyl)phenoxy)-N-(1H-indol-4-yl)acetamide of the following formula: 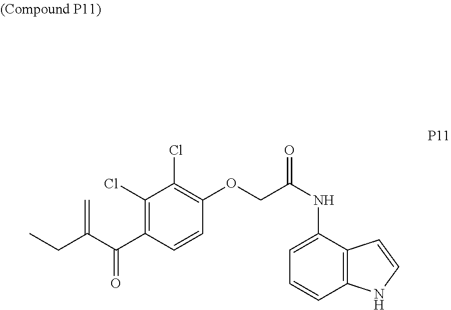 [SMILES: C=C(CC)C(=O)c1ccc(OCC(=O)Nc2cccc3[nH]ccc23)c(Cl)c1Cl]
2-(2,3-dichloro-4-(2-methylenebutanoyl)phenoxy)-N-(1H-indol-5-yl)acetamide of the following formula: 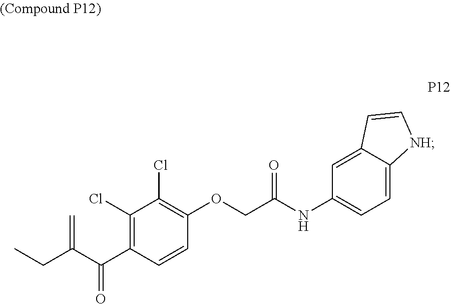 [SMILES: C=C(CC)C(=O)c1ccc(OCC(=O)Nc2ccc3[nH]ccc3c2)c(Cl)c1Cl]
2-(2,3-dichloro-4-(2-methylenebutanoyl)phenoxy)-N-(1H-indol-6-yl)acetamide of the following formula: 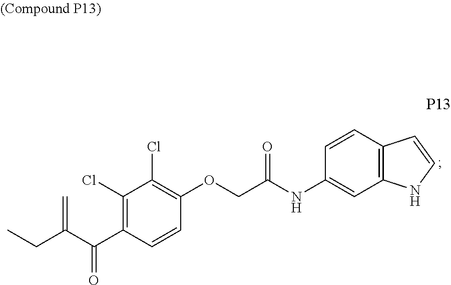 [SMILES: C=C(CC)C(=O)c1ccc(OCC(=O)Nc2ccc3cc[nH]c3c2)c(Cl)c1Cl]
2-(2,3-dichloro-4-(2-methylenebutanoyl)phenoxy)-N-(1-methyl-1H-indazol-4-yl)acetamide of the following formula: 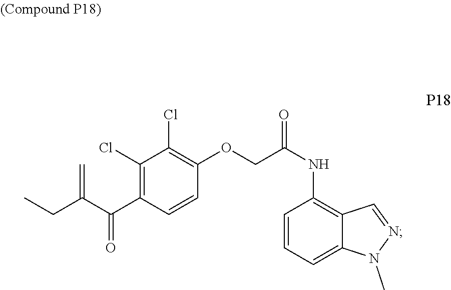 [SMILES: C=C(CC)C(=O)c1ccc(OCC(=O)Nc2cccc3c2cnn3C)c(Cl)c1Cl]
2-(2,3-dichloro-4-(2-methylenebutanoyl)phenoxy)-N-(1-methyl-1H-indazol-6-yl)acetamide of the following formula: 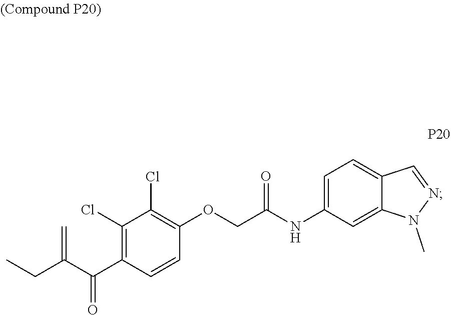 [SMILES: C=C(CC)C(=O)c1ccc(OCC(=O)Nc2ccc3cnn(C)c3c2)c(Cl)c1Cl]
2-(2,3-dichloro-4-(2-methylenebutanoyl)phenoxy)-N-(1-methyl-1H-indazol-7-yl)acetamide of the following formula: 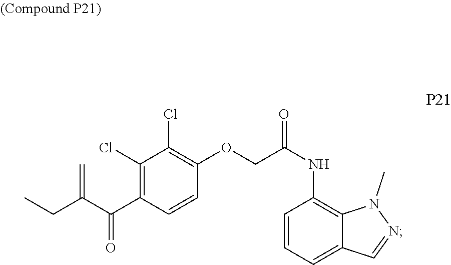 [SMILES: C=C(CC)C(=O)c1ccc(OCC(=O)Nc2cccc3cnn(C)c23)c(Cl)c1Cl]
1-(4-(2-(1H-indazol-1-yl)-2-oxoethoxy)-2,3-dichlorophenyl)-2-methylenebutan-1-one of the following formula: 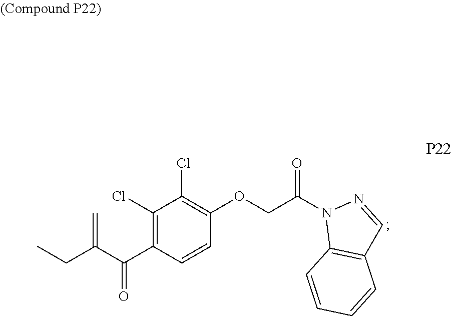 [SMILES: C=C(CC)C(=O)c1ccc(OCC(=O)n2ncc3ccccc32)c(Cl)c1Cl]
2-(2,3-dichloro-4-(2-methylenebutanoyl)phenoxy)-N-(1H-indazol-5-yl)acetamide of the following formula: 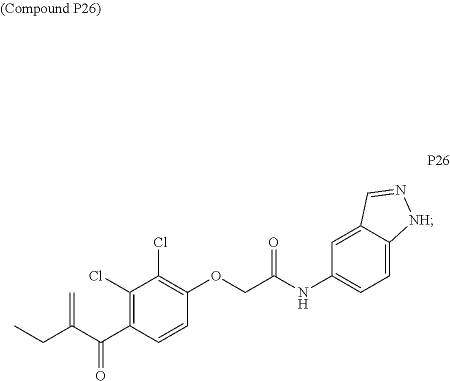 [SMILES: C=C(CC)C(=O)c1ccc(OCC(=O)Nc2ccc3[nH]ncc3c2)c(Cl)c1Cl]
2-(2,3-dichloro-4-(2-methylenebutanoyl)phenoxy)-N-(2-methyl-2H-indazol-6-yl)acetamide of the following formula: 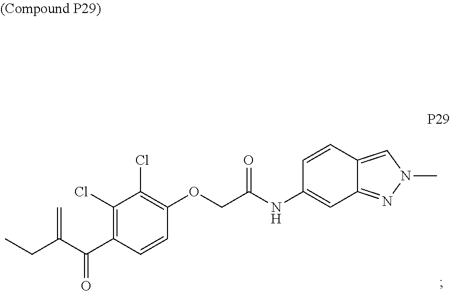 [SMILES: C=C(CC)C(=O)c1ccc(OCC(=O)Nc2ccc3cn(C)nc3c2)c(Cl)c1Cl]
2-(2,3-dichloro-4-(2-methylenebutanoyl)phenoxy)-N-(2-methyl-2H-indazol-7-yl)acetamide of the following formula: 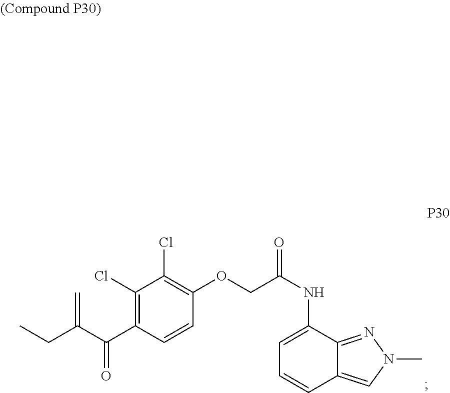 [SMILES: C=C(CC)C(=O)c1ccc(OCC(=O)Nc2cccc3cn(C)nc23)c(Cl)c1Cl]
2-(2,3-dichloro-4-(2-methylenebutanoyl)phenoxy)-N-(2-methyl-2H-indazol-4-yl)acetamide of the following formula: 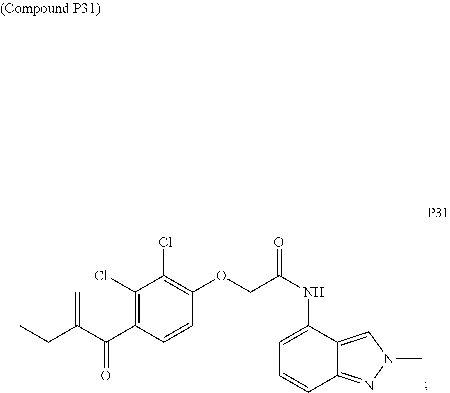 [SMILES: C=C(CC)C(=O)c1ccc(OCC(=O)Nc2cccc3nn(C)cc23)c(Cl)c1Cl]
Tert-butyl 5-(2-(2,3-dichloro-4-(2-methylenebutanoyl)phenoxy)acetamido)-1H-indazole-1-carboxylate of the following formula: 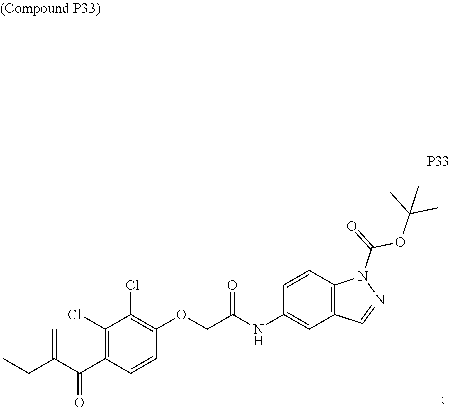 [SMILES: C=C(CC)C(=O)c1ccc(OCC(=O)Nc2ccc3c(cnn3C(=O)OC(C)(C)C)c2)c(Cl)c1Cl]
2-(2,3-dichloro-4-(2-methylenebutanoyl)phenoxy)-N-(1-(prop-2-yn-1-yl)-1H-indazol-4-yl)acetamide of the following formula: 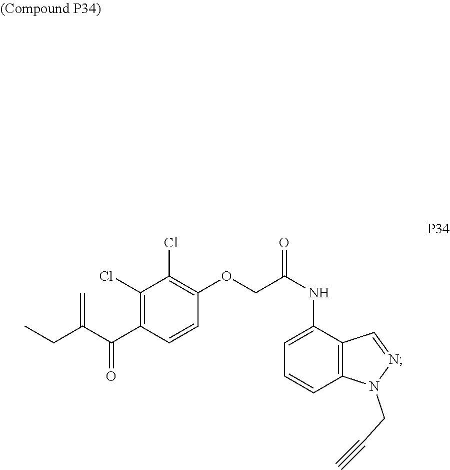 [SMILES: C#CCn1ncc2c(NC(=O)COc3ccc(C(=O)C(=C)CC)c(Cl)c3Cl)cccc21]
Tert-butyl (2-(2-(2,3-dichloro-4-(2-methylenebutanoyl)phenoxy)acetamido)ethyl)-carbamate of the following formula: 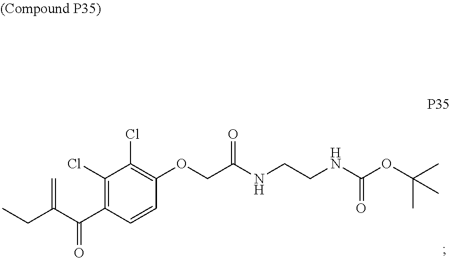 [SMILES: C=C(CC)C(=O)c1ccc(OCC(=O)NCCNC(=O)OC(C)(C)C)c(Cl)c1Cl]
Tert-butyl (1-(2-(2,3-dichloro-4-(2-methylenebutanoyl)phenoxy)acetyl)piperidin-4-yl)carbamate of the following formula: 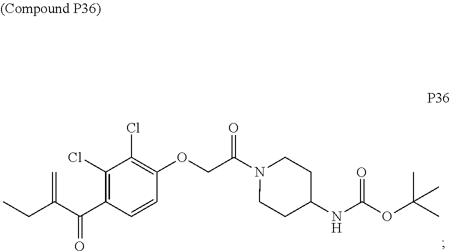 [SMILES: C=C(CC)C(=O)c1ccc(OCC(=O)N2CCC(NC(=O)OC(C)(C)C)CC2)c(Cl)c1Cl]
N-(2-chloroethyl)-2-(2,3-dichloro-4-(2-methylenebutanoyl)phenoxy)acetamide of the following formula: 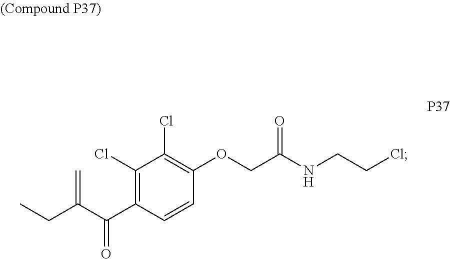 [SMILES: C=C(CC)C(=O)c1ccc(OCC(=O)NCCCl)c(Cl)c1Cl]
Tert-butyl 4-(2-(2,3-dichloro-4-(2-methylenebutanoyl)phenoxy)acetyl)piperazine-1-carboxylate of the following formula: 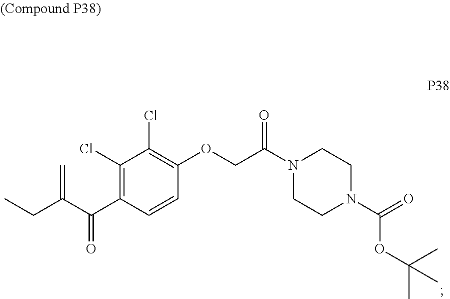 [SMILES: C=C(CC)C(=O)c1ccc(OCC(=O)N2CCN(C(=O)OC(C)(C)C)CC2)c(Cl)c1Cl]
N-butyl-2-(2,3-dichloro-4-(2-methylenebutanoyl)phenoxy)acetamide of the following formula: 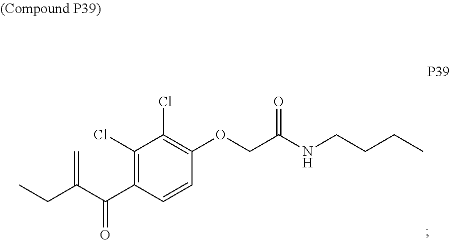 [SMILES: C=C(CC)C(=O)c1ccc(OCC(=O)NCCCC)c(Cl)c1Cl]
2-(2,3-dichloro-4-(2-methylenebutanoyl)phenoxy)-N-((1-methyl-1H-indazol-5-yl)methyl) acetamide of the following formula: 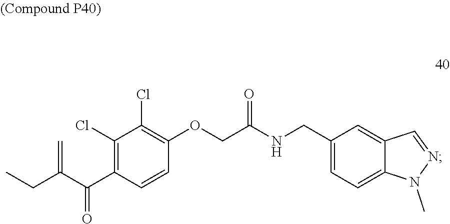 [SMILES: C=C(CC)C(=O)c1ccc(OCC(=O)NCc2ccc3c(cnn3C)c2)c(Cl)c1Cl]
1-(2,3-dichloro-4-(2-(5-methyl-1H-indazol-1-yl)-2-oxoethoxy)phenyl)-2-methylenebutan-1-one of the following formula: 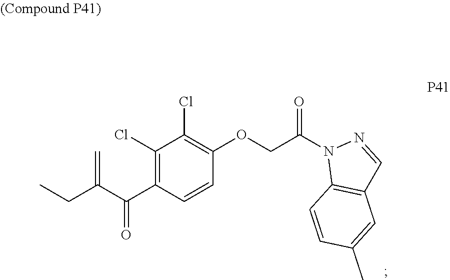 [SMILES: C=C(CC)C(=O)c1ccc(OCC(=O)n2ncc3cc(C)ccc32)c(Cl)c1Cl]
1-(2,3-dichloro-4-(2-oxo-2-(1H-pyrazolo[4,3-b]pyridin-1-yl)ethoxy)phenyl)-2-methylenebutan-1-one of the following formula: 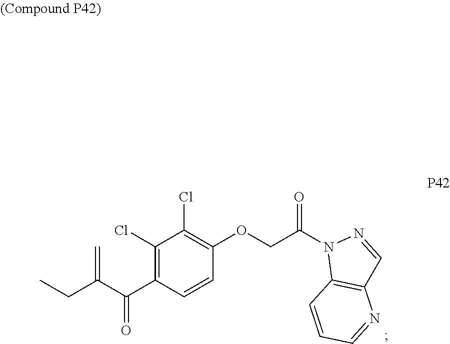 [SMILES: C=C(CC)C(=O)c1ccc(OCC(=O)n2ncc3ncccc32)c(Cl)c1Cl]
2-(2,3-dichloro-4-(2-methylenebutanoyl)phenoxy)-N-(1-methyl-1/-benzo[d]imidazol-5-yl)acetamide of the following formula:

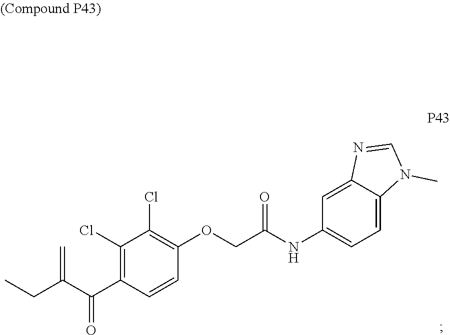 [SMILES: C=C(CC)C(=O)c1ccc(OCC(=O)Nc2ccc3c(c2)ncn3C)c(Cl)c1Cl]

3-The compound according to claim 1 for application as a drug.

4-The compound according to claim 3, for application and as a drug for treatment of COVID-19 disease.

5-The compound according to claim 3, for application and as a drug for treatment of pathologies caused by β-coronaviruses.

6-The compound according to claim 3, for application in vitro for inhibiting SARS-COV-2 replication.

7-The compound according to claim 3, for application in vitro for inhibiting M^{pro}protease.

8-A use of method comprising providing a compound of the formulae (I) or (II) labelled according to claim 3 as a research tool, especially for the identification of molecules capable of interacting with the active site of the MPRO protease.

9-The compound according to claim 1, for use as an active principle in pharmaceutical compositions.

10-The compound according to claim 1, for use in combination with other drugs in pharmaceutical compositions.

11-The compound according to claim 1, wherein W is a hydrogen or chlorine atom and Z is a chlorine, fluorine, bromine atom or hydroxyl.

12-The compound according to claim 1, wherein V and X are nitrogen atoms and Y is a carbon or nitrogen atom.
